(19) United States
(12) Patent Application Publication
(10) Pub. No.: US 2021/0403866 A1
(43) Pub. Date: Dec. 30, 2021

(54) ENHANCED DENDRITIC CELL IMMUNE ACTIVATION BY COMBINED INHIBITION OF NR2F6 WITH CANNIBIDIOL

(71) Applicant: Regen Biopharma, Inc., Medina, MN (US)

(72) Inventors: Thomas Ichim, San Diego, CA (US); David Koos, La Mesa, CA (US)

(73) Assignee: Regen Biopharma, Inc., Medina, MN (US)

(21) Appl. No.: 17/035,955

(22) Filed: Sep. 29, 2020

Related U.S. Application Data
(60) Provisional application No. 62/882,931, filed on Aug. 5, 2019.

(51) Int. Cl. C12N 5/0784; A61K 35/15; C12N 15/113

(52) U.S. Cl. CPC C12N 5/0639; A61K 35/15; C12N 2310/14; C12N 2501/999; C12N 2310/11; C12N 15/1138

(57) ABSTRACT

Cancer associated inflammation has been reported to act as an immune suppressive mechanism through augmentation of myeloid suppressor cells, as well as downregulation of T cell receptor (TCR) signaling through the cleavage of the CD3 zeta chain. The teachings herein show the effects of concurrent anti-inflammatory treatment of dendritic cells (DC) using cannabidiol (CBD) together with inhibition of an immunological checkpoint, that is, silencing of NR2F6. Mixed lymphocyte cultures are used to increased potency of dendritic cells to stimulate allogeneic T cell proliferative and cytokine responses by the combination of NR2F6 gene silencing together with CBD. CBD alone appeared to inhibit DC maturation and allogestimulatory activity. These data support the utilization of small molecule inhibitors of NR2F6 together with CBD as combination therapies for immune modulation.

### CROSS-REFERENCE TO RELATED APPLICATIONS

This application claims the benefit of priority to U.S. Provisional Application No. 62/882,931, filed Aug. 5, 2019, the contents of which are incorporated herein by reference.

### BACKGROUND

Cancer immunotherapy has developed into a new branch of oncology, offering substantial benefit to patients with various stages of cancer, which previously had no treatment options. For example, the clinical entry of checkpoint inhibitors has resulted in successful treatment of metastatic melanoma, lung cancer, and kidney cancer. Despite these advances, a majority of patients still do no respond to checkpoint inhibitors. Numerous reasons are cited including the immune suppressive environment of the tumor. In order to establish an effective vaccine for active specific immunotherapy, several obstacles must be overcome. Firstly, due to the poor specificities of tumor antigens or large tumor burdens, and patients' overall immunodeficiency caused by pre-chemotherapy as well as other factors, it is very difficult to induce the immune response of active specific immunotherapy which aims at tumor antigens. Secondly, because the antigenicity of tumor cells (particularly the immunogenicity of human spontaneously occurred tumors) is generally very weak, it may be not easy for the vaccine immunization to induce an adequate immune response to shrink the tumor. Thirdly, because of the heterogeneity in the expression of the tumor antigen, most patients need to be immunized by various antigens simultaneously. Fourthly, one of the main problems of developing a cancer vaccine is that it is hard to build an ideal animal model which is similar to the patients' clinical situation.

There have been studies showing that passive specific immunotherapy, namely transferring sensitized tumor-specific T lymphocytes, can treat the tumor of experimental animals effectively (confirmed in the late 60s and the early 70s), many issues including insufficient sensitized T lymphocytes, the difficulty of rejection reaction to be induced due to the weakness of antigens of the spontaneous tumors, and the incompatibility of sensitized T lymphocytes obtained from animal experiments for humans, hinder the study and the treatment of sensitized T lymphocytes. The biggest problem for the application of adoptive cellular immune therapy is how to obtain sufficient sensitized tumor-specific T lymphocytes.

Active non-specific immunity can stimulate the immune system by non-specific stimulation to enhance non-specific immune reactions; it can improve the immune reactions to existing tumors. The majority of active non-specific immunotherapy applied on humans in the past is not successful and most of them have not been used today. Almost all of the active non-specific treatments for patients who had advanced cancers are unsuccessful.

There is another form of immunotherapy termed passive non-specific immunotherapy, such as LAK/IL-2 and TIL/TDAK (tumor-derived activated killing cells) therapies, mainly aims at killing tumor cells by means of infusing their autologous or allogenic non-specific effectors. However, the effectiveness of these therapies are limited and need to be improved. For example, patients' insufficient autologous LAK precursor cells, along with slow amplification and limited efficacy, all lead to the ineffectiveness of the treatment. Moreover, patients usually cannot tolerate it due to serious side effects brought upon by repeated applications of high-dose rIL-2. The biggest disadvantages of TDAK therapy (which are more obvious than that of LAK therapy) are that it is time-consuming, laborious, and costly. The activity of TIL depends on the type, size and the extent of necrosis of the tumor, and not all tumors are infiltrated by lymphocytes. As a matter of fact, in most tumors it is difficult to obtain autologous tumor-specific TIL. The proliferative capability and anti-tumor activity of TIL will be reduced along with prolonged couture time. In addition, apoptosis can be found in some of the cells in the course of the cell culture. The TIL obtained from the tumors that produce immunosuppressive factors may be unable to proliferate, similar to the TIL obtained from metastatic tumors. In view of this, how to achieve these high tumor-reactive lymphocyte subsets and amplify them in vitro becomes the important issue of TIL/TDAK.

One promising means of treating cancer is the utilization of dendritic cells that are programmed outside of the body in order to induce T cell activation. Such dendritic cells are usually grown from patient peripheral blood and/or mobilized bone marrow progenitors. Unfortunately, means of growing such dendritic cells are limited in that the dendritic cell is a proliferatively terminal cell and thus does not expand ex vivo. Accordingly, it is necessary to identify means of enhancing dendritic cell activity in vitro so as to generated cells with enhanced potency of activating host immunity.

### SUMMARY

Preferred embodiments are directed to methods of augmenting immune stimulatory activity of dendritic cells comprising: a) providing a dendritic cell population; b) treating said dendritic cell population with cannabidiol; and c) suppressing expression and/or activity of NR2F6 in said dendritic cells.

Preferably said immune stimulatory activity is ability of dendritic cells to activate T cells, cytokine production, ability to convert to memory cells, acquisition of cytotoxic activity, ability to activate antibody production from B cells, ability to induce isotype switching, ability to induce maturation of dendritic cells, ability to induce NK cells to possess an enhancement of cytotoxic activity.

In certain embodiments, the dendritic cells are myeloid derived dendritic cells or lymphoid derived dendritic cells.

Preferably the cannabidiol possesses anti-inflammatory properties.

Preferably said dendritic cell is further activated with a toll like receptor agonist such as TLR-1, including Pam3CSK4.

Alternatively said toll like receptor is TLR-2, and said activator of TLR-2 is HKLM.

According to further embodiments said toll like receptor is TLR-3.

According to further embodiments said activator of TLR-3 is Poly:IC.

According to further embodiments said toll like receptor is TLR-4.

According to further embodiments said activator of TLR-4 is LPS.

According to further embodiments said activator of TLR-4 is Buprenorphine.

According to further embodiments said activator of TLR-4 is Carbamazepine.

According to further embodiments said activator of TLR-4 is Fentanyl.

According to further embodiments said activator of TLR-4 is Levorphanol.

According to further embodiments said activator of TLR-4 is Methadone.

According to further embodiments said activator of TLR-4 is Morphine.

According to further embodiments said activator of TLR-4 is Oxcarbazepine.

According to further embodiments said activator of TLR-4 is Oxycodone.

According to further embodiments said activator of TLR-4 is Pethidine.

According to further embodiments said activator of TLR-4 is Glucuronoxylomannan from Cryptococcus.

According to further embodiments said activator of TLR-4 is Morphine-3-glucuronide.

According to further embodiments said activator of TLR-4 is lipoteichoic acid.

According to further embodiments said activator of TLR-4 is β-defensin 2.

According to further embodiments said activator of TLR-4 is small molecular weight hyaluronic acid.

According to further embodiments said activator of TLR-4 is fibronectin EDA.

According to further embodiments said activator of TLR-4 is snapin.

According to further embodiments said activator of TLR-4 is tenascin C.

According to further embodiments said toll like receptor is TLR-5.

According to further embodiments said activator of TLR-5 is flagellin.

According to further embodiments said toll like receptor is TLR-6.

According to further embodiments said activator of TLR-6 is FSL-1.

According to further embodiments said toll like receptor is TLR-7.

According to further embodiments said activator of TLR-7 is imiquimod.

According to further embodiments said toll like receptor of TLR-8.

According to further embodiments said activator of TLR8 is ssRNA40/LyoVec.

According to further embodiments said toll like receptor of TLR-9.

According to further embodiments said activator of TLR-9 is a CpG oligonucleotide.

According to further embodiments said activator of TLR-9 is ODN2006.

According to further embodiments said activator of TLR-9 is Agatolimod.

According to further embodiments suppression of NR2F6 is achieved by administration of an antisense oligonucleotide molecule capable of suppressing expression of NR2F6.

According to further embodiments suppression of said NR2F6 is mediated by activation of RNAse H.

According to further embodiments said suppression of NR2F6 is achieved by treatment of said dendritic cells with nucleic acids capable of inducing the processes of RNA interference.

According to further embodiments said nucleic acids capable of inducing the process of RNA interference are short double stranded RNA.

According to further embodiments said nucleic acids capable of inducing the process of RNA interference are short hairpin RNA.

According to further embodiments said nucleic acids capable of inducing the process of RNA interference are ribozymes.

According to further embodiments said nucleic acids capable of inducing the process of RNA interference are hammerhead ribozymes.

### BRIEF DESCRIPTION OF THE DRAWINGS

FIG. 1 is a bar graph showing a dose dependent suppression of T cell activation for a Mixed Lymphocyte Reaction (MLR). Increased amounts of cannabidiol added to dendritic led to higher suppression of T cell activation.

FIG. 2 is a bar graph showing a dose dependent increase in allostimulatory activity for an MLR was performed as in example 1. Increased amounts of cannabidiol added to dendritic cells silenced for NR2F6 led to an increase in allostimulatory activity.

FIG. 3 is a line graph showing tumor inhibition of a) saline, b) dendritic cells that are NR2F6 silenced, c) dendritic cells treated with CBD, and d) NR2F6 silenced dendritic cells+CBD. As shown, mice treated with dendritic cells that were conditioned with cannabidiol and gene silencing for NR2F6 possessed the highest degree of tumor inhibition.

### DESCRIPTION OF THE INVENTION

The term “antibody,” as used herein, refers to an immunoglobulin molecule which specifically binds with an antigen. Antibodies can be intact immunoglobulins derived from natural sources or from recombinant sources and can be immunoreactive portions of intact immunoglobulins. Antibodies are typically tetramers of immunoglobulin molecules. The antibodies in the present invention may exist in a variety of forms including, for example, polyclonal antibodies, monoclonal antibodies, Fv, Fab and F(ab).sub.2, as well as single chain antibodies and humanized antibodies. The term “antibody fragment” refers to a portion of an intact antibody and refers to the antigenic determining variable regions of an intact antibody. Examples of antibody fragments include, but are not limited to, Fab, Fab′, F(ab′)2, and Fv fragments, linear antibodies, scFv antibodies, and multispecific antibodies formed from antibody fragments.

An “antibody heavy chain,” as used herein, refers to the larger of the two types of polypeptide chains present in all antibody molecules in their naturally occurring conformations.

An “antibody light chain,” as used herein, refers to the smaller of the two types of polypeptide chains present in all antibody molecules in their naturally occurring conformations. .kappa. and .lamda. light chains refer to the two major antibody light chain isotypes.

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

The term “antigen” or “Ag” as used herein is defined as a molecule that provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full length nucleotide sequence of a gene. It is readily apparent that the present invention includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

The term “anti-tumor effect” as used herein, refers to a biological effect which can be manifested by a decrease in tumor volume, a decrease in the number of tumor cells, a decrease in the number of metastases, an increase in life expectancy, or amelioration of various physiological symptoms associated with the cancerous condition. An “anti-tumor effect” can also be manifested by the ability of the peptides, polynucleotides, cells and antibodies of the invention in prevention of the occurrence of tumor in the first place.

The term “auto-antigen” means, in accordance with the present invention, any self-antigen which is mistakenly recognized by the immune system as being foreign. Auto-antigens comprise, but are not limited to, cellular proteins, phosphoproteins, cellular surface proteins, cellular lipids, nucleic acids, glycoproteins, including cell surface receptors.

As used herein, the term “autologous” is meant to refer to any material derived from the same individual to which it is later to be re-introduced into the individual.

“Allogeneic” refers to a graft derived from a different animal of the same species. The term “cancer” as used herein is defined as disease characterized by the rapid and uncontrolled growth of aberrant cells. Cancer cells can spread locally or through the bloodstream and lymphatic system to other parts of the body. Examples of various cancers include but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer and the like. Other specific types of cancer include cinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiennoid carcinoma, carcinoma epitheliale adenoides, exophytic carcinoma, carcinoma ex ulcere, carcinoma fibrosum, gelatiniform carcinoma, gelatinous carcinoma, giant cell carcinoma, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrmcous carcinoma, carcinoma villosum, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypemephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, naspharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, and carcinoma scroti, The term “sarcoma” generally refers to a tumor which is made up of a substance like the embryonic connective tissue and is generally composed of closely packed cells embedded in a fibrillar, heterogeneous, or homogeneous substance. Sarcomas include, chondrosarcoma, fibrosarcoma, lymphosarcoma, melanosarcoma, myxosarcoma, osteosarcoma, endometrial sarcoma, stromal sarcoma, Ewing's sarcoma, fascial sarcoma, fibroblastic sarcoma, giant cell sarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilns' tumor sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, and telangiectaltic sarcoma. Additional exemplary neoplasias include, for example, Hodgkin's Disease, Non-Hodgkin's Lymphoma, multiple myeloma, neuroblastoma, breast cancer, ovarian cancer, lung cancer, rhabdomyosarcoma, primary thrombocytosis, primary macroglobulinemia, small-cell lung tumors, primary brain tumors, stomach cancer, colon cancer, malignant pancreatic insulanoma, malignant carcinoid, premalignant skin lesions, testicular cancer, lymphomas, thyroid cancer, neuroblastoma, esophageal cancer, genitourinary tract cancer, malignant hypercalcemia, cervical cancer, endometrial cancer, and adrenal cortical cancer.

In some particular embodiments of the invention, the cancer treated is a melanoma. The term “melanoma” is taken to mean a tumor arising from the melanocytic system of the skin and other organs. Melanomas include, for example, Harding-Passey melanoma, juvenile melanoma, lentigo maligna melanoma, malignant melanoma, acral-lentiginous melanoma, amelanotic melanoma, benign juvenile melanoma, Cloudman's melanoma, S91 melanoma, nodular melanoma subungal melanoma, and superficial spreading melanoma.

“Costimulatory ligand” as used herein, includes a molecule on an antigen presenting cell that specifically binds a cognate co-stimulatory molecule on a T cell. Binding of the costimulatory ligand provides a signal that mediates a T cell response, including, but not limited to, proliferation, activation, differentiation, and the like. A costimulatory ligand induces a signal that is in addition to the primary signal provided by a stimulatory molecule, for instance, by binding of a T cell receptor (TCR)/CD3 complex with a major histocompatibility complex (MHC) molecule loaded with peptide. A co-stimulatory ligand can include, but is not limited to, CD7, B7-1 (CD80), B7-2 (CD86), programmed death (PD) L1, PD-L2, 4-1BB ligand, OX40 ligand, inducible costimulatory ligand (ICOS-L), intercellular adhesion molecule (ICAM), CD30 ligand, CD40, CD70, CD83, human leukocyte antigen G (HLA-G), MHC class I chain-related protein A (MICA), MHC class I chain-related protein B (MICB), herpes virus entry mediator (HVEM), lymphotoxin beta receptor, 3/TR6, immunoglobulin-like transcript (ILT) 3, ILT4, an agonist or antibody that binds Toll ligand receptor and a ligand that specifically binds with B7-H3. A co-stimulatory ligand includes, without limitation, an antibody that specifically binds with a co-stimulatory molecule present on a T cell, such as, but not limited to, CD27, CD28, 4-1BB, OX40, CD30, CD40, PD-1, ICOS, lymphocyte function-associated antigen-1 (LFA-1), CD2, CD7, tumor necrosis factor superfamily member 14 (TNFSF14 or LIGHT), natural killer cell receptor C (NKG2C), B7-H3, and a ligand that specifically binds with CD83.

The term “CD137” refers to a TNFR-family member with costimulatory function. CD137 is also called 4-1BB or TNFSFR9. It was originally identified as an inducible molecule expressed on activated mouse and human CD8+ and CD4+ T-cells. CD137 signaling regulates T-cell proliferation and survival, particularly within the T-cell memory pool and can upregulate Bcl-X.sub.L anti-apoptotic protein expression and supports CD8+ T-cell expansion.

“Cytokine,” as used herein, refers to a non-antibody protein that is released by one cell in response to contact with a specific antigen, wherein the cytokine interacts with a second cell to mediate a response in the second cell. A cytokine can be endogenously expressed by a cell or administered to a subject. Cytokines may be released by immune cells, including macrophages, B cells, T cells, and mast cells to propagate an immune response. Cytokines can induce various responses in the recipient cell. Cytokines can include homeostatic cytokines, chemokines, pro-inflammatory cytokines, effectors, and acute-phase proteins. For example, homeostatic cytokines, including interleukin (IL) 7 and IL-15, promote immune cell survival and proliferation, and pro-inflammatory cytokines can promote an inflammatory response. Examples of homeostatic cytokines include, but are not limited to, IL-2, IL-4, IL-5, IL-7, IL-10, IL-12p40, IL-12p′70, IL-15, and interferon (IFN) gamma. Examples of pro-inflammatory cytokines include, but are not limited to, IL-1a, IL-1b, IL-6, IL-13, IL-17a, tumor necrosis factor (TNF)-alpha, TNF-beta, fibroblast growth factor (FGF) 2, granulocyte macrophage colony-stimulating factor (GM-CSF), soluble intercellular adhesion molecule 1 (sICAM-1), soluble vascular adhesion molecule 1 (sVCAM-1), vascular endothelial growth factor (VEGF), VEGF-C, VEGF-D, and placental growth factor (PLGF). Examples of effectors include, but are not limited to, granzyme A, granzyme B, soluble Fas ligand (sFasL), and perforin. Examples of acute phase-proteins include, but are not limited to, C-reactive protein (CRP) and serum amyloid A (SAA).

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

An “effective amount” as used herein, means an amount which provides a therapeutic or prophylactic benefit.

The term “immunoglobulin” or “Ig,” as used herein is defined as a class of proteins, which function as antibodies. Antibodies expressed by B cells are sometimes referred to as the BCR (B cell receptor) or antigen receptor. The five members included in this class of proteins are IgA, IgG, IgM, IgD, and IgE. IgA is the primary antibody that is present in body secretions, such as saliva, tears, breast milk, gastrointestinal secretions and mucus secretions of the respiratory and genitourinary tracts. IgG is the most common circulating antibody. IgM is the main immunoglobulin produced in the primary immune response in most subjects. It is the most efficient immunoglobulin in agglutination, complement fixation, and other antibody responses, and is important in defense against bacteria and viruses. IgD is the immunoglobulin that has no known antibody function, but may serve as an antigen receptor. IgE is the immunoglobulin that mediates immediate hypersensitivity by causing release of mediators from mast cells and basophils upon exposure to allergen.

As used herein, an “instructional material” includes a publication, a recording, a diagram, or any other medium of expression which can be used to communicate the usefulness of the compositions and methods of the invention. The instructional material of the kit of the invention may, for example, be affixed to a container which contains the nucleic acid, peptide, and/or composition of the invention or be shipped together with a container which contains the nucleic acid, peptide, and/or composition. Alternatively, the instructional material may be shipped separately from the container with the intention that the instructional material and the compound be used cooperatively by the recipient.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

The term “lymphocyte” means T cells, B cells, NK cells, and Lymphokine Activated Killer (LAK) cells. T-lymphocytes possess T-cell receptors, B-lymphocytes, possess B cell receptors and produce antibodies, Tumor Infiltrating Lymphocytes (TIL) are isolated from tumors and possess some degree of reactivity towards the tumor, cytotoxic T lymphocytes (CTL) are lymphocytes of the CD8 lineage usually and possess ability to kill cells through perforin and/or granzymes. CTL isolation means are described in numerous references including U.S. Pat. Nos. 6,805,861 and 6,531,451. Any one lymphocyte produces one type of TCR or antibody. Each TCR or antibody has specificity for one particular epitope, or antigen binding site, on its cognate antigen. Specific TCRs or antibodies are encoded by genes that are formed from the rearrangement of DNA in a lymphocyte stem cell that encodes the constant (“C”), joining (“J”), variable (“V”) regions, and possibly diversity (“D”) regions of the TCR or antibody. Mammals typically possess one-hundred thousand to one-hundred million lymphocytes of different specificities. Upon stimulation of lymphocytes by an antigen, those lymphocytes specific for the antigen undergo clonal amplification. T lymphocytes are formed in the bone marrow, migrate to and mature in the thymus and then enter the peripheral blood and lymphatic circulation. T lymphocytes are subdivided into three distinct types of cells: helper T cells, suppressor T cells, and cytotoxic T cells. T lymphocytes, unlike B lymphocytes, do not produce antibody molecules, but express a heterodimeric cell surface receptor that recognizes peptide fragments of antigenic proteins that are attached to proteins of the major histocompatibility complex (MHC) and expressed on the surfaces of target cells. T lymphocytes include tumor-infiltrating lymphocytes. Cytotoxic T lymphocytes (CTL) are well known in the art and are typically of the CD3+, CD8+, CD4− phenotype. They typically lyse cells that display fragments of foreign antigens associated with class I MHC molecules on their cell surfaces. CTL typically recognize normal cells expressing antigens after infection by viruses or other pathogens; and tumor cells that have undergone transformation and are expressing mutated proteins or are over-expressing normal proteins. Natural Killer (NK) cells are well known in the art. NK cells are a subset of lymphocytes active in the immune system and representing an average 15% of mononuclear cells in human peripheral blood. Among the surface markers used to identify human NK cells is a receptor binding with low affinity to the Fc fragment of IgG antibodies, such as Fc-.gamma. receptor III or CD16 antigen. NK cells have been demonstrated to play an important role in vivo in the defense against tumors, tumor metastases, virus infection, and to regulate normal and malignant hematopoiesis. Lymphokine-activated killer (LAK) cells are well known in the art and are a cytotoxic population of cells which are capable of lysing autologous tumor cells and NK-cell resistant tumor cell lines. Precursors of LAK cells belong to the subpopulation of “null” lymphocytes that bear neither T nor B cell surface markers. In the human these precursor cells are widely found in peripheral blood, lymph nodes, bone marrow and the thoracic duct. Purification of LAK cells, and their generation are described in U.S. Pat. Nos. 5,002,879, 4,849,329 and 4,690,915.

The term “modulating,” as used herein, is meant mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, preferably, a human.

The term “overexpressed” tumor antigen or “overexpression” of the tumor antigen is intended to indicate an abnormal level of expression of the tumor antigen in a cell from a disease area like a solid tumor within a specific tissue or organ of the patient relative to the level of expression in a normal cell from that tissue or organ. Patients having solid tumors or a hematological malignancy characterized by overexpression of the tumor antigen can be determined by standard assays known in the art.

“Parenteral” administration of an immunogenic composition includes, e.g., subcutaneous (s.c.), intravenous (i.v.), intramuscular (i.m.), or intrasternal injection, or infusion techniques.

The terms “patient,” “subject,” “individual,” and the like are used interchangeably herein, and refer to any animal, such as a mammal, or cells thereof whether in vitro or in situ, amenable to the methods described herein. In certain non-limiting embodiments, the patient, subject or individual is a human.

By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

By the term “stimulation,” is meant a primary response induced by binding of a stimulatory molecule (e.g., a TCR/CD3 complex) with its cognate ligand thereby mediating a signal transduction event, such as, but not limited to, signal transduction via the TCR/CD3 complex. Stimulation can mediate altered expression of certain molecules, such as downregulation of TGF-.beta, and/or reorganization of cytoskeletal structures, and the like.

A “stimulatory molecule,” as the term is used herein, means a molecule on a T cell that specifically binds with a cognate stimulatory ligand present on an antigen presenting cell.

A “stimulatory ligand,” as used herein, means a ligand that when present on an antigen presenting cell (e.g., an artificial APC (1), a dendritic cell, a B-cell, and the like) can specifically bind with a cognate binding partner (referred to herein as a “stimulatory molecule”) on a T cell, thereby mediating a primary response by the T cell, including, but not limited to, activation, initiation of an immune response, proliferation, and the like. Stimulatory ligands are well-known in the art and encompass, inter alia, an MHC Class I molecule loaded with a peptide, an anti-CD3 antibody, a superagonist anti-CD28 antibody, and a superagonist anti-CD2 antibody.

The term “subject” is intended to include living organisms in which an immune response can be elicited (e.g., mammals). Examples of subjects include humans, dogs, cats, mice, rats, and transgenic species thereof.

As used herein, a “substantially purified” cell is a cell that is essentially free of other cell types. A substantially purified cell also refers to a cell which has been separated from other cell types with which it is normally associated in its naturally occurring state. In some instances, a population of substantially purified cells refers to a homogenous population of cells. In other instances, this term refers simply to cell that have been separated from the cells with which they are naturally associated in their natural state. In some embodiments, the cells are cultured in vitro. In other embodiments, the cells are not cultured in vitro.

The term “therapeutic” as used herein means a treatment and/or prophylaxis. A therapeutic effect is obtained by suppression, remission, or eradication of a disease state.

The term “therapeutically effective amount” refers to the amount of the subject compound that will elicit the biological or medical response of a tissue, system, or subject that is being sought by the researcher, veterinarian, medical doctor or other clinician. The term “therapeutically effective amount” includes that amount of a compound that, when administered, is sufficient to prevent development of, or alleviate to some extent, one or more of the signs or symptoms of the disorder or disease being treated. The therapeutically effective amount will vary depending on the compound, the disease and its severity and the age, weight, etc., of the subject to be treated.

To “treat” a disease as the term is used herein, means to reduce the frequency or severity of at least one sign or symptom of a disease or disorder experienced by a subject.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

The invention describes the unique, useful, and previously unknown synergy between inhibition of NR2F6 and cannabidiol for activating dendritic cells. NR2F6 is an orphan receptor which has been demonstrated to be found in various cancer cells, as well as to inhibit activation of immune responses. Originally described as a 1643-bp cDNA that encodes the mouse Ear-2 (original name of NR2F6) orphan nuclear receptor isolated from a pituitary cell library. The predicted 389-aa mouse Ear-2 protein bears significant homology to the known human Ear-2 protein especially in the DNA-binding domain (2). Studies describing the molecular biology (3-9), the impact on cancer (10), and on immunotherapy (11), are incorporated by reference to assist one of skill in the art in understanding basic features of NR2F6.

In one embodiment dendritic cells are generated from leukocytes of patients by leukopheresis. Numerous means of leukopheresis are known in the art. In one example, a Frenius Device (Fresenius Com.Tec) is utilized with the use of the MNC program, at approximately 1500 rpm, and with a P1Y kit. The plasma pump flow rates are adjusted to approximately 50 mL/min. Various anticoagulants may be used, for example ACD-A. The Inlet/ACD Ratio may be ranged from approximately 10:1 to 16:1. In one embodiment approximately 150 mL of blood is processed. The leukopheresis product is subsequently used for initiation of dendritic cell culture. In order to generatesa peripheral blood mononuclear cells from leukopheresis product, mononuclear cells are isolated by the Ficoll-Hypaque density gradient centrifugation. Monocytes are then enriched by the Percoll hyperosmotic density gradient centrifugation followed by two hours of adherence to the plate culture. Cells are then centrifuged at 500 g to separate the different cell populations. Adherent monocytes are cultured for 7 days in 6-well plates at 2×106 cells/mL RMPI medium with 1% penicillin/streptomycin, 2 mM L-glutamine, 10% of autologous, 50 ng/mL GM-CSF and 30 ng/mL IL-4. On day 6 immature dendritic cells are pulsed with tumor antigens, as well as treated with cannabidiol and NR2F6 siRNA. Pulsing may be performed by incubation of lysates with dendritic cells, or may be generated by fusion of immature dendritic cells with cancer cells. Means of generating hybridomas or cellular fusion products are known in the art and include electrical pulse mediated fusion, or stimulation of cellular fusion by treatment with polyethelyne glycol. On day 7, the immature DCs are then induced to differentiate into mature DCs by culturing for 48 hours with 30 ng/mL interferon gamma (IFN-γ). During the course of generating DC for clinical purposes, microbiologic monitoring tests are performed at the beginning of the culture, on the fifth day and at the time of cell freezing for further use or prior to release of the dendritic cells. Administration of pulsed dendritic cells is utilized as a polyvalent vaccine, whereas subsequent to administration antibody or t cell responses are assessed for induction of antigen specificity, peptides corresponding to immune response stimulated are used for further immunization to focus the immune response. Generation of dendritic cells are described in the following publications for various types of cancers and incorporated by reference: melanoma (12-63), soft tissue sarcoma (64), thyroid (65-67), glioma (68-89), multiple myeloma, (90-98), lymphoma (99-101), leukemia (102-109), as well as liver (110-115), lung (116-129), ovarian (130-133), and pancreatic cancer (134-136).

In one embodiment, the utilization of adjuvants is performed in order to endow an activation state of dendritic cells, wherein cannabidiol is concurrently administered with the adjuvant in order to allow for enhanced dendritic cell activation in the presence of suppressed inflammation, in which said cannabidiol acts to suppress said inflammation. Adjuvants that may be used together with cannabidiol for the purpose of the invention are compounds which enhance activation of dendritic cells and/or Th1 immunity. Known adjuvants in the art that may be utilized include; Cationic liposome-DNA complex JVRS-100, aluminum hydroxide, aluminum phosphate vaccine, aluminum potassium sulfate adjuvant, Alhydrogel, ISCOM(s), Freund's Complete Adjuvant, Freund's Incomplete Adjuvant, CpG DNA Vaccine Adjuvant, Cholera toxin, Cholera toxin B subunit liposomes, Saponin, DDA, Squalene-based Adjuvants, Etx B subunit, IL-12, LTK63 Vaccine Mutant Adjuvant, TiterMax Gold Adjuvant, Ribi Vaccine Adjuvant, Montanide ISA 720 Adjuvant, Corynebacterium-derived P40 Vaccine Adjuvant, MPL™ Adjuvant, ASO4, AS02, Lipopolysaccharide Vaccine Adjuvant, Muramyl Dipeptide Adjuvant, CRL1005, Killed Corynebacterium parvum Vaccine Adjuvant, Montanide ISA 51, Bordetella pertussis component Vaccine Adjuvant, Cationic Liposomal Vaccine Adjuvant, Adamantylamide Dipeptide Vaccine Adjuvant, Arlacel A, VSA-3 Adjuvant, Aluminum vaccine adjuvant, Polygen Vaccine Adjuvant, Adjumer™, Algal Glucan, Bay R1005, Theramide®, thalidomide, Stearyl Tyrosine, Specol, Algammulin, Avridine®, Calcium Phosphate Gel, CTA1-DD gene fusion protein, DOC/Alum Complex, Gamma Inulin, Gerbu Adjuvant, GM-CSF, GMDP, Recombinant hIFN-gamma/Interferon-g, Interleukin-1β, Interleukin-2, Interleukin-7, Sclavo peptide, Rehydragel LV, Rehydragel HPA, Loxoribine, MF59, MTP-PE Liposomes, Murametide, Murapalmitine, D-Murapalmitine, NAGO, Non-Ionic Surfactant Vesicles, PMMA, Protein Cochleates, QS-21, SPT (Antigen Formulation), nanoemulsion vaccine adjuvant, AS03, Quil-A vaccine adjuvant, RC529 vaccine adjuvant, LTR192G Vaccine Adjuvant, E. coli heat-labile toxin, LT, amorphous aluminum hydroxyphosphate sulfate adjuvant, Calcium phosphate vaccine adjuvant, Montanide Incomplete Seppic Adjuvant, Imiquimod, Resiquimod, AF03, Flagellin, Poly(I:C), ISCOMATRIX®, Abisco-100 vaccine adjuvant, Albumin-heparin microparticles vaccine adjuvant, AS-2 vaccine adjuvant, B7-2 vaccine adjuvant, DHEA vaccine adjuvant, Immunoliposomes Containing Antibodies to Costimulatory Molecules, SAF-1, Sendai Proteoliposomes, Sendai-containing Lipid Matrices, Threonyl muramyl dipeptide (TMDP), Ty Particles vaccine adjuvant, Bupivacaine vaccine adjuvant, DL-PGL (Polyester poly (DL-lactide-co-glycolide)) vaccine (137)E112K of Cholera Toxin mCT-E112K, and Matrix-S.

The cancer vaccine formulation may be utilized in conjunction with known adjuvants in order to induce an immune response that is Th1 or Th17-like, and which will inhibit the proliferation of endothelial cells in the recipient. Such adjuvant compounds are known in the art to boost the activity of the immune system and are now under study as possible adjuvants, particularly for vaccine therapies. Some of the most commonly studied adjuvants are listed below, but many more are under development. For example, Levamisole, a drug originally used against parasitic infections, has recently been found to improve survival rates among people with colorectal cancer when used together with some chemotherapy drugs (138-144). It is often used as an immunotherapy adjuvant because it can activate T lymphocytes (145-147). Additionally, the compound has been demonstrated to induce maturation of dendritic cells, further supporting an immune modulatory role (148). Levamisole is now used routinely for people with some stages of colorectal cancer and is being tested in clinical trials as a treatment for other types of cancer. Additionally, it has been shown to augment efficacy of other immunotherapeutic agents such as interferon (149,150). Aluminum hydroxide (alum) is one of the most common adjuvants used in clinical trials for cancer vaccines. It is already used in vaccines against several infectious agents, including the hepatitis B virus. Bacille Calmette-Guerin (BCG) is a bacterium that is related to the bacterium that causes tuberculosis. The effect of BCG infection on the immune system makes this bacterium useful as a form of anticancer immunotherapy (151). BCG was one of the earliest immunotherapies used against cancer, either alone, or in combination with other therapies such as hormonal, chemotherapy or radiotherapy (152-160). It is FDA approved as a routine treatment for superficial bladder cancer. Its usefulness in other cancers as a nonspecific adjuvant is also being tested or has demonstrated therapeutic effects (161-169). Researchers are looking at injecting BCG to give an added stimuli to the immune system when using chemotherapy, radiation therapy, or other types of immunotherapy. Thus in various embodiments of the current invention, one of skill in the art is directed towards references which have utilized BCG as an adjuvant for other therapies for concentrations and dosing regimens that would apply to the current invention for elicitation of immunity towards proliferating endothelial cells. Incomplete Freund's Adjuvant (IFA) is given together with some experimental therapies to help stimulate the immune system and to increase the immune response to cancer vaccines, both protein and peptide in part by providing a localization factor for T cells (170-178). IFA is a liquid consisting of an emulsifier in white mineral oil. Another vaccine adjuvant useful for the present invention is interferon alpha, which has been demonstrated to augment NK cell activity, as well as to promote T cell activation and survival (179). QS-21 is a relatively new immune stimulant made from a plant extract that increases the immune response to vaccines used against melanoma. DETOX is another relatively new adjuvant. It is made from parts of the cell walls of bacteria and a kind of fat. It is used with various immunotherapies to stimulate the immune system. Keyhole limpet hemocyanin (KLH) is another adjuvant used to boost the effectiveness of cancer vaccine therapies. It is extracted from a type of sea mollusc. Dinitrophenyl (DNP) is a hapten/small molecule that can attach to tumor antigens and cause an enhanced immune response. It is used to modify tumor cells in certain cancer vaccines. In some embodiments the mixture of NR2F6 silencing and cannabidiol is utilized to enhance tumor vaccine efficacy.

In some embodiments of the invention, immunity to tumor antigens is induced by pulsing dendritic cells with said tumor antigens these antigens can be chosen from known groups of tumor antigens such as ERG (180-190), WT1 (109,191-249), BCR-ABL (250-264), Ras-mutant (265), MUC1 (135,266-286), ETV6-AML (287), LMP2 (288-295), p53 non-mutant (296-298), MYC-N, survivin, androgen receptor, RhoC, cyclin B1, EGFRvIII, EphA2, B cell or T cell idiotype, ML-IAP, BORIS, hTERT, PLAC1, HPV E6, HPV E7, OY-TES1, Her2/neu, PAX3, NY-BR-1, p53 mutant, MAGE A3, EpCAM, polysialic Acid, AFP, PAXS, NY-ESO1, sperm protein 17, GD3, Fucosyl GM1, mesothelin, PSMA, GD2, MAGE A1, sLe(x), HMWMAA, CYP1B1, sperm fibrous sheath protein, B7H3, TRP-2, AKAP-4, XAGE 1, CEA, Tn, GloboH, SSX2, RGSS, SART3, gp100, MelanA/MART1, Tyrosinase, GM3 ganglioside, Proteinase 3 (PR1), Page4, STn, Carbonic anhydrase IX, PSCA, Legumain, MAD-CT-1 (protamin2), PSA, Tie 2, MAD-CT2, PAP, PDGFR-beta, NA17, VEGFR2, FAP, LCK, Fos-related antigen, LCK, FAP.

In some embodiments of the invention, the utilization of various chemotherapies is performed in order to enhance antigenicity of the cancer through augmentation of antigen expression. The ability of chemotherapy to enhance expression of tumor antigens was elegantly demonstrated in a study in which gemcitabine (GEM), a standard therapeutic drug for PC, was examined for the regulation of WT1 expression and the sensitizing effect on PC cells with WT1-specific antitumor immune response. Expression of WT1 was examined by quantitative PCR, immunoblot analysis, and confocal microscopy. Antigenic peptide of WT1 presented on HLA class I molecules was detected by mass spectrometry. WT1-specific T-cell receptor gene-transduced human T cells were used as effecter T cells for the analysis of cytotoxic activity. GEM treatment of human MIAPaCa2 PC cells enhanced WT1 mRNA levels, and this increase is associated with nuclear factor kappa B activation. Tumor tissue from GEM-treated MIAPaCa2-bearing SCID mice also showed an increase in WT1 mRNA. Some human PC cell lines other than MIAPaCa2 showed up-regulation of WT1 mRNA levels following GEM treatment. GEM treatment shifted WT1 protein from the nucleus to the cytoplasm, which may promote proteasomal processing of WT1 protein and generation of antigenic peptide. In fact, presentation of HLA-A*2402-restricted antigenic peptide of WT1 (CMTWNQMNL) increased in GEM-treated MIAPaCa2 cells relative to untreated cells. WT1-specific cytotoxic T cells killed MIAPaCa2 cells treated with an optimal dose of GEM more efficiently than untreated MIAPaCa2 cells. GEM enhanced WT1 expression in human PC cells and sensitized PC cells with WT1-specific T-cell-mediated antitumor immune response (299). In other embodiments, the use of agents that affect DNA methylation are utilized to enhance expression of tumor antigens. The ability of such agents to induce upregulation of tumor antigens was illustrated in a study in which investigators treated the murine WT1-transfected C1498 (mWT1-C1498) with increasing doses of decitabine (DAC) and azacitidine (AZA) to analyze their effects on transgene reactivation. DAC and AZA decreased the number of viable cells in a dose- or time-dependent manner. Quantification of WT1 mRNA level was analyzed by real-time polymerase chain reaction after mWT1-C1498 treated with increasing dose of HMA. DAC treatment for 48 h induced 1.4-, 14.6-, and 15.5-fold increment of WT1 mRNA level, compared to untreated sample, at 0.1, 1, and 10 μM, respectively. Further increment of WT1 expression in the presence of 1 and 10 μM DAC was evident at 72 h. AZA treatment also induced up-regulation of mRNA, but not to the same degree as with DAC treatment. The correlation between the incremental increases in WT1 mRNA by DAC was confirmed by Western blot and concomitant down-regulation of WT1 promoter methylation was revealed. The in vitro data show that HMA can induce reactivation of WT1 transgene and that DAC is more effective, at least in mWT1-C1498 cells, which suggests that the combination of DAC and mWT1-C1498 can be used for the development of the experimental model of HMA-combined WT1 immunotherapy targeting leukemia (300).

In other embodiments of the invention, tumor antigens may be administered using oral delivery means, and subsequently immunity can be primed with dendritic cells that are concurrently treated with cannabidiol and NR2F6 silenced. For example, methods of generating oral vaccines have been described such as in this study in which investigators constructed an oral cancer vaccine using a recombinant Bifidobacterium longum displaying WT1 protein. B. longum 420 was orally administered into mice inoculated with WT1-expressing tumor cells for 4 weeks to examine anti-tumor effects. To analyze the WT1-specific cellular immune responses to oral B. longum 420, mice splenocytes were isolated and cytokine production and cytotoxic activities were determined. Oral administrations of B. longum 420 significantly inhibited WT1-expressing tumor growth and prolonged survival in mice. Immunohistochemical study and immunological assays revealed that B. longum 420 substantially induced tumor infiltration of CD4⁺ T and CD8⁺ T cells, systemic WT1-specific cytokine production, and cytotoxic activity mediated by WT1-epitope specific cytotoxic T lymphocytes, with no apparent adverse effects. The oral cancer vaccine safely induced WT1-specific cellular immunity via activation of the gut mucosal immune system and achieved therapeutic efficacy with several practical advantages over existing non-oral vaccines (301).

### EXAMPLES

### Suppression of Mixed Lymphocyte Reaction by Cannabidiol

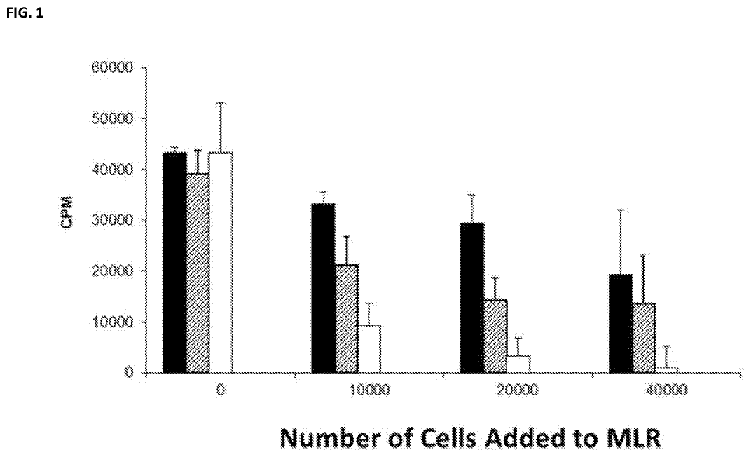

Dendritic cells where added to allogeneic T cells in 96 well plates, wherein allogeneic T cells where added at 40,000 cells per plate and the indicated number of dendritic cells added. Culture was performed for 96 hours with tritiated thymidine added at the last 12 hours of culture at a concentration of 1 microcurie per well. Black bars indicate no cannabidiol added to dendritic cells, striped bars indicate 1 micro Molar and white indicate 2 micromolar. A dose dependent suppression of T cell activation was observed. Results are shown in graph, FIG. 1.

### Example 2: Gene Silencing of NR2F6 Enhances Ability to Stimulate Mixed Lymphocyte Reaction

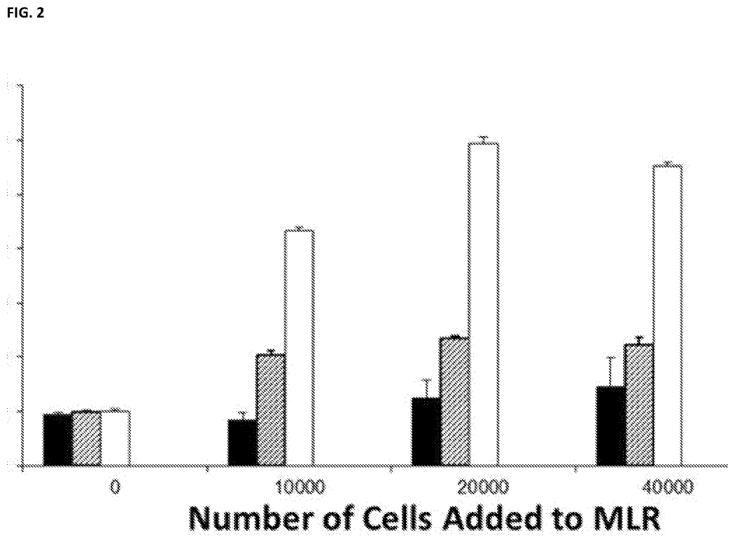

MLR was performed as in example 1. All cells where silenced for NR2F6. An increase in allostimulatory activity was observed. Results are shown in graph, FIG. 2.

### Example 3: Inhibition of Tumor Growth

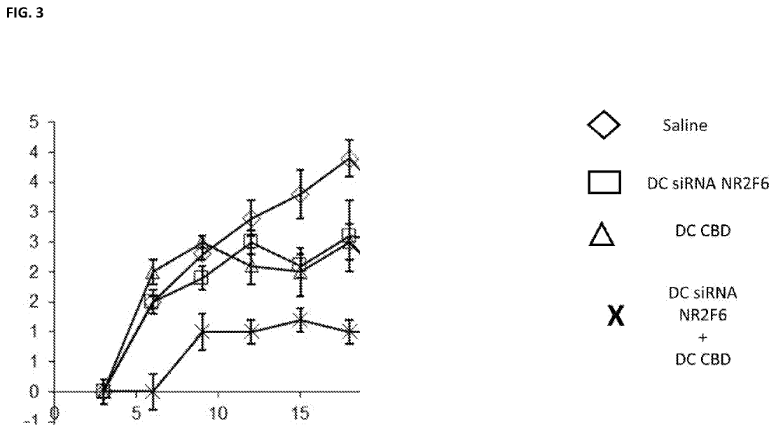

Lewis lung carcinoma cells where implanted into syngeneic mice at a concentration of 500,000 cells per mouse and tumor growth was observed. As seen below mice treated with dendritic cells that were conditioned with cannabidiol (2 microMolar) and gene silencing for NR2F6 possessed the highest degree of tumor inhibition. Results are shown in graph, FIG. 3.

### REFERENCES

1. Maus, M. V., Thomas, A. K., Leonard, D. G., Allman, D., Addya, K., Schlienger, K., Riley, J. L., and June, C. H. (2002) Ex vivo expansion of polyclonal and antigen-specific cytotoxic T lymphocytes by artificial APCs expressing ligands for the T-cell receptor, CD28 and 4-1BB. Nat Biotechnol 20, 143-148
2. Barnhart, K. M., and Mellon, P. L. (1994) The sequence of a murine cDNA encoding Ear-2, a nuclear orphan receptor. Gene 142, 313-314
3. Islam, T. C., and Toftgard, R. (1994) Nuclear orphan receptor-binding retinoic acid response elements in keratinocytes. Biochem Biophys Res Commun 203, 545-552
4. Chu, K., and Zingg, H. H. (1997) The nuclear orphan receptors COUP-TFII and Ear-2 act as silencers of the human oxytocin gene promoter. J Mol Endocrinol 19, 163-172
5. Chu, K., Boutin, J. M., Breton, C., and Zingg, H. H. (1998) Nuclear orphan receptors COUP-TFII and Ear-2: presence in oxytocin-producing uterine cells and functional interaction with the oxytocin gene promoter. Mol Cell Endocrinol 137, 145-154
6. Avram, D., Ishmael, J. E., Nevrivy, D. J., Peterson, V. J., Lee, S. H., Dowell, P., and Leid, M. (1999) Heterodimeric interactions between chicken ovalbumin upstream promoter-transcription factor family members ARP1 and ear2. J Biol Chem 274, 14331-14336
7. Zhang, Y., and Dufau, M. L. (2000) Nuclear orphan receptors regulate transcription of the gene for the human luteinizing hormone receptor. J Biol Chem 275, 2763-2770
8. Zhu, X. G., Park, K. S., Kaneshige, M., Bhat, M. K., Zhu, Q., Mariash, C. N., McPhie, P., and Cheng, S. Y. (2000) The orphan nuclear receptor Ear-2 is a negative coregulator for thyroid hormone nuclear receptor function. Mol Cell Biol 20, 2604-2618
9. Liu, X., Huang, X., and Sigmund, C. D. (2003) Identification of a nuclear orphan receptor (Ear2) as a negative regulator of renin gene transcription. Circ Res 92, 1033-1040
10. Yang, C., Yu, B., Zhou, D., and Chen, S. (2002) Regulation of aromatase promoter activity in human breast tissue by nuclear receptors. Oncogene 21, 2854-2863
11. Klepsch, V., Hermann-Kleiter, N., and Baier, G. (2016) Beyond CTLA-4 and PD-1: Orphan nuclear receptor NR2F6 as T cell signaling switch and emerging target in cancer immunotherapy. Immunol Lett 178, 31-36
12. Nestle, F. O., Alijagic, S., Gilliet, M., Sun, Y., Grabbe, S., Dummer, R., Burg, G., and Schadendorf, D. (1998) Vaccination of melanoma patients with peptide- or tumor lysate-pulsed dendritic cells. Nature medicine 4, 328-332
13. Chakraborty, N. G., Sporn, J. R., Tortora, A. F., Kurtzman, S. H., Yamase, H., Ergin, M. T., and Mukherji, B. (1998) Immunization with a tumor-cell-lysate-loaded autologous-antigen-presenting-cell-based vaccine in melanoma. Cancer immunology, immunotherapy: CII 47, 58-64
14. Wang, F., Bade, E., Kuniyoshi, C., Spears, L., Jeffery, G., Marty, V., Groshen, S., and Weber, J. (1999) Phase I trial of a MART-1 peptide vaccine with incomplete Freund's adjuvant for resected high-risk melanoma. Clinical cancer research: an official journal of the American Association for Cancer Research 5, 2756-2765
15. Thurner, B., Haendle, I., Roder, C., Dieckmann, D., Keikavoussi, P., Jonuleit, H., Bender, A., Maczek, C., Schreiner, D., von den Driesch, P., Brocker, E. B., Steinman, R. M., Enk, A., Kampgen, E., and Schuler, G. (1999) Vaccination with mage-3A1 peptide-pulsed mature, monocyte-derived dendritic cells expands specific cytotoxic T cells and induces regression of some metastases in advanced stage IV melanoma. The Journal of experimental medicine 190, 1669-1678
16. Thomas, R., Chambers, M., Boytar, R., Barker, K., Cavanagh, L. L., MacFadyen, S., Smithers, M., Jenkins, M., and Andersen, J. (1999) Immature human monocyte-derived dendritic cells migrate rapidly to draining lymph nodes after intradermal injection for melanoma immunotherapy. Melanoma research 9, 474-481
17. Mackensen, A., Herbst, B., Chen, J. L., Kohler, G., Noppen, C., Herr, W., Spagnoli, G. C., Cerundolo, V., and Lindemann, A. (2000) Phase I study in melanoma patients of a vaccine with peptide-pulsed dendritic cells generated in vitro from CD34(+) hematopoietic progenitor cells. International journal of cancer. Journal international du cancer 86, 385-392
18. Panelli, M. C., Wunderlich, J., Jeffries, J., Wang, E., Mixon, A., Rosenberg, S. A., and Marincola, F. M. (2000) Phase 1 study in patients with metastatic melanoma of immunization with dendritic cells presenting epitopes derived from the melanoma-associated antigens MART-1 and gp100. Journal of immunotherapy 23, 487-498
19. Schuler-Thurner, B., Dieckmann, D., Keikavoussi, P., Bender, A., Maczek, C., Jonuleit, H., Roder, C., Haendle, I., Leisgang, W., Dunbar, R., Cerundolo, V., von Den Driesch, P., Knop, J., Brocker, E. B., Enk, A., Kampgen, E., and Schuler, G. (2000) Mage-3 and influenza-matrix peptide-specific cytotoxic T cells are inducible in terminal stage HLA-A2.1+melanoma patients by mature monocyte-derived dendritic cells. Journal of immunology 165, 3492-3496
20. Lau, R., Wang, F., Jeffery, G., Marty, V., Kuniyoshi, J., Bade, E., Ryback, M. E., and Weber, J. (2001) Phase I trial of intravenous peptide-pulsed dendritic cells in patients with metastatic melanoma. Journal of immunotherapy 24, 66-78
21. Banchereau, J., Palucka, A. K., Dhodapkar, M., Burkeholder, S., Taquet, N., Rolland, A., Taquet, S., Coquery, S., Wittkowski, K. M., Bhardwaj, N., Pineiro, L., Steinman, R., and Fay, J. (2001) Immune and clinical responses in patients with metastatic melanoma to CD34(+) progenitor-derived dendritic cell vaccine. Cancer research 61, 6451-6458
22. Schuler-Thurner, B., Schultz, E. S., Berger, T. G., Weinlich, G., Ebner, S., Woerl, P., Bender, A., Feuerstein, B., Fritsch, P. O., Romani, N., and Schuler, G. (2002) Rapid induction of tumor-specific type 1 T helper cells in metastatic melanoma patients by vaccination with mature, cryopreserved, peptide-loaded monocyte-derived dendritic cells. The Journal of experimental medicine 195, 1279-1288
23. Palucka, A. K., Dhodapkar, M. V., Paczesny, S., Burkeholder, S., Wittkowski, K. M., Steinman, R. M., Fay, J., and Banchereau, J. (2003) Single injection of CD34+ progenitor-derived dendritic cell vaccine can lead to induction of T-cell immunity in patients with stage IV melanoma. Journal of immunotherapy 26, 432-439
24. Bedrosian, I., Mick, R., Xu, S., Nisenbaum, H., Faries, M., Zhang, P., Cohen, P. A., Koski, G., and Czerniecki, B. J. (2003) Intranodal administration of peptide-pulsed mature dendritic cell vaccines results in superior CD8+ T-cell function in melanoma patients. Journal of clinical oncology: official journal of the American Society of Clinical Oncology 21, 3826-3835
25. Slingluff, C. L., Jr., Petroni, G. R., Yamshchikov, G. V., Barnd, D. L., Eastham, S., Galavotti, H., Patterson, J. W., Deacon, D. H., Hibbitts, S., Teates, D., Neese, P. Y., Grosh, W. W., Chianese-Bullock, K. A., Woodson, E. M., Wiernasz, C. J., Merrill, P., Gibson, J., Ross, M., and Engelhard, V. H. (2003) Clinical and immunologic results of a randomized phase II trial of vaccination using four melanoma peptides either administered in granulocyte-macrophage colony-stimulating factor in adjuvant or pulsed on dendritic cells. Journal of clinical oncology: official journal of the American Society of Clinical Oncology 21, 4016-4026
26. Hersey, P., Menzies, S. W., Halliday, G. M., Nguyen, T., Farrelly, M. L., DeSilva, C., and Lett, M. (2004) Phase I/II study of treatment with dendritic cell vaccines in patients with disseminated melanoma. Cancer immunology, immunotherapy: CII 53, 125-134
27. Vilella, R., Benitez, D., Mila, J., Lozano, M., Vilana, R., Pomes, J., Tomas, X., Costa, J., Vilalta, A., Malvehy, J., Puig, S., Mellado, B., Marti, R., and Castel, T. (2004) Pilot study of treatment of biochemotherapy-refractory stage IV melanoma patients with autologous dendritic cells pulsed with a heterologous melanoma cell line lysate. Cancer immunology, immunotherapy: CII 53, 651-658
28. Palucka, A. K., Connolly, J., Ueno, H., Kohl, J., Paczesny, S., Dhodapkar, M., Fay, J., and Banchereau, J. (2005) Spontaneous proliferation and type 2 cytokine secretion by CD4+ T cells in patients with metastatic melanoma vaccinated with antigen-pulsed dendritic cells. Journal of clinical immunology 25, 288-295
29. Banchereau, J., Ueno, H., Dhodapkar, M., Connolly, J., Finholt, J. P., Klechevsky, E., Blanck, J. P., Johnston, D. A., Palucka, A. K., and Fay, J. (2005) Immune and clinical outcomes in patients with stage IV melanoma vaccinated with peptide-pulsed dendritic cells derived from CD34+ progenitors and activated with type I interferon. Journal of immunotherapy 28, 505-516
30. Trakatelli, M., Toungouz, M., Blocklet, D., Dodoo, Y., Gordower, L., Laporte, M., Vereecken, P., Sales, F., Mortier, L., Mazouz, N., Lambermont, M., Goldman, S., Coulie, P., Goldman, M., and Velu, T. (2006) A new dendritic cell vaccine generated with interleukin-3 and interferon-beta induces CD8+ T cell responses against NA17-A2 tumor peptide in melanoma patients. Cancer immunology, immunotherapy: CII 55, 469-474
31. Salcedo, M., Bercovici, N., Taylor, R., Vereecken, P., Massicard, S., Duriau, D., Vernel-Pauillac, F., Boyer, A., Baron-Bodo, V., Mallard, E., Bartholeyns, J., Goxe, B., Latour, N., Leroy, S., Prigent, D., Martiat, P., Sales, F., Laporte, M., Bruyns, C., Romet-Lemonne, J. L., Abastado, J. P., Lehmann, F., and Velu, T. (2006) Vaccination of melanoma patients using dendritic cells loaded with an allogeneic tumor cell lysate. Cancer immunology, immunotherapy: CII 55, 819-829
32. Linette, G. P., Zhang, D., Hodi, F. S., Jonasch, E. P., Longerich, S., Stowell, C. P., Webb, I. J., Daley, H., Soiffer, R. J., Cheung, A. M., Eapen, S. G., Fee, S. V., Rubin, K. M., Sober, A. J., and Haluska, F. G. (2005) Immunization using autologous dendritic cells pulsed with the melanoma-associated antigen gp100-derived G280-9V peptide elicits CD8+ immunity. Clinical cancer research: an official journal of the American Association for Cancer Research 11, 7692-7699
33. Escobar, A., Lopez, M., Serrano, A., Ramirez, M., Perez, C., Aguirre, A., Gonzalez, R., Alfaro, J., Larrondo, M., Fodor, M., Ferrada, C., and Salazar-Onfray, F. (2005) Dendritic cell immunizations alone or combined with low doses of interleukin-2 induce specific immune responses in melanoma patients. Clinical and experimental immunology 142, 555-568
34. Tuettenberg, A., Becker, C., Huter, E., Knop, J., Enk, A. H., and Jonuleit, H. (2006) Induction of strong and persistent MelanA/MART-1-specific immune responses by adjuvant dendritic cell-based vaccination of stage II melanoma patients. International journal of cancer. Journal international du cancer 118, 2617-2627
35. Schadendorf, D., Ugurel, S., Schuler-Thurner, B., Nestle, F. O., Enk, A., Brocker, E. B., Grabbe, S., Rittgen, W., Edler, L., Sucker, A., Zimpfer-Rechner, C., Berger, T., Kamarashev, J., Burg, G., Jonuleit, H., Tuttenberg, A., Becker, J. C., Keikavoussi, P., Kampgen, E., Schuler, G., and DeCOG, D. C. s. g. o. t. (2006) Dacarbazine (DTIC) versus vaccination with autologous peptide-pulsed dendritic cells (DC) in first-line treatment of patients with metastatic melanoma: a randomized phase III trial of the DC study group of the DeCOG. Annals of oncology: official journal of the European Society for Medical Oncology/ESMO 17, 563-570
36. Di Pucchio, T., Pilla, L., Capone, I., Ferrantini, M., Montefiore, E., Urbani, F., Patuzzo, R., Pennacchioli, E., Santinami, M., Cova, A., Sovena, G., Arienti, F., Lombardo, C., Lombardi, A., Caporaso, P., D'Atri, S., Marchetti, P., Bonmassar, E., Parmiani, G., Belardelli, F., and Rivoltini, L. (2006) Immunization of stage IV melanoma patients with Melan-A/MART-1 and gp100 peptides plus IFN-alpha results in the activation of specific CD8(+) T cells and monocyte/dendritic cell precursors. Cancer research 66, 4943-4951
37. Nakai, N., Asai, J., Ueda, E., Takenaka, H., Katoh, N., and Kishimoto, S. (2006) Vaccination of Japanese patients with advanced melanoma with peptide, tumor lysate or both peptide and tumor lysate-pulsed mature, monocyte-derived dendritic cells. The Journal of dermatology 33, 462-472
38. Palucka, A. K., Ueno, H., Connolly, J., Kerneis-Norvell, F., Blanck, J. P., Johnston, D. A., Fay, J., and Banchereau, J. (2006) Dendritic cells loaded with killed allogeneic melanoma cells can induce objective clinical responses and MART-1 specific CD8+ T-cell immunity. Journal of immunotherapy 29, 545-557
39. Lesimple, T., Neidhard, E. M., Vignard, V., Lefeuvre, C., Adamski, H., Labarriere, N., Carsin, A., Monnier, D., Collet, B., Clapisson, G., Birebent, B., Philip, I., Toujas, L., Chokri, M., and Quillien, V. (2006) Immunologic and clinical effects of injecting mature peptide-loaded dendritic cells by intralymphatic and intranodal routes in metastatic melanoma patients. Clinical cancer research: an official journal of the American Association for Cancer Research 12, 7380-7388
40. Guo, J., Zhu, J., Sheng, X., Wang, X., Qu, L., Han, Y., Liu, Y., Zhang, H., Huo, L., Zhang, S., Lin, B., and Yang, Z. (2007) Intratumoral injection of dendritic cells in combination with local hyperthermia induces systemic antitumor effect in patients with advanced melanoma. International journal of cancer. Journal international du cancer 120, 2418-2425
41. O'Rourke, M. G., Johnson, M. K., Lanagan, C. M., See, J. L., O'Connor, L. E., Slater, G. J., Thomas, D., Lopez, J. A., Martinez, N. R., Ellem, K. A., and Schmidt, C. W. (2007) Dendritic cell immunotherapy for stage IV melanoma. Melanoma research 17, 316-322
42. Bercovici, N., Haicheur, N., Massicard, S., Vernel-Pauillac, F., Adotevi, O., Landais, D., Gorin, I., Robert, C., Prince, H. M., Grob, J. J., Leccia, M. T., Lesimple, T., Wijdenes, J., Bartholeyns, J., Fridman, W. H., Salcedo, M., Ferries, E., and Tartour, E. (2008) Analysis and characterization of antitumor T-cell response after administration of dendritic cells loaded with allogeneic tumor lysate to metastatic melanoma patients. Journal of immunotherapy 31, 101-112
43. Hersey, P., Halliday, G. M., Farrelly, M. L., DeSilva, C., Lett, M., and Menzies, S. W. (2008) Phase I/II study of treatment with matured dendritic cells with or without low dose IL-2 in patients with disseminated melanoma. Cancer immunology, immunotherapy: CII 57, 1039-1051
44. von Euw, E. M., Barrio, M. M., Furman, D., Levy, E. M., Bianchini, M., Peguillet, I., Lantz, O., Vellice, A., Kohan, A., Chacon, M., Yee, C., Wainstok, R., and Mordoh, J. (2008) A phase I clinical study of vaccination of melanoma patients with dendritic cells loaded with allogeneic apoptotic/necrotic melanoma cells. Analysis of toxicity and immune response to the vaccine and of IL-10-1082 promoter genotype as predictor of disease progression. Journal of translational medicine 6, 6
45. Carrasco, J., Van Pel, A., Neyns, B., Lethe, B., Brasseur, F., Renkvist, N., van der Bruggen, P., van Baren, N., Paulus, R., Thielemans, K., Boon, T., and Godelaine, D. (2008) Vaccination of a melanoma patient with mature dendritic cells pulsed with MAGE-3 peptides triggers the activity of nonvaccine anti-tumor cells. Journal of immunology 180, 3585-3593
46. Redman, B. G., Chang, A. E., Whitfield, J., Esper, P., Jiang, G., Braun, T., Roessler, B., and Mule, J. J. (2008) Phase Ib trial assessing autologous, tumor-pulsed dendritic cells as a vaccine administered with or without IL-2 in patients with metastatic melanoma. Journal of immunotherapy 31, 591-598
47. Daud, A. I., Mirza, N., Lenox, B., Andrews, S., Urbas, P., Gao, G. X., Lee, J. H., Sondak, V. K., Riker, A. I., Deconti, R. C., and Gabrilovich, D. (2008) Phenotypic and functional analysis of dendritic cells and clinical outcome in patients with high-risk melanoma treated with adjuvant granulocyte macrophage colony-stimulating factor. Journal of clinical oncology: official journal of the American Society of Clinical Oncology 26, 3235-3241
48. Engell-Noerregaard, L., Hansen, T. H., Andersen, M. H., Thor Straten, P., and Svane, I. M. (2009) Review of clinical studies on dendritic cell-based vaccination of patients with malignant melanoma: assessment of correlation between clinical response and vaccine parameters. Cancer immunology, immunotherapy: CII 58, 1-14
49. Nakai, N., Katoh, N., Germeraad, W. T., Kishida, T., Ueda, E., Takenaka, H., Mazda, O., and Kishimoto, S. (2009) Immunohistological analysis of peptide-induced delayed-type hypersensitivity in advanced melanoma patients treated with melanoma antigen-pulsed mature monocyte-derived dendritic cell vaccination. Journal of dermatological science 53, 40-47
50. Dillman, R. O., Selvan, S. R., Schiltz, P. M., McClay, E. F., Barth, N. M., DePriest, C., de Leon, C., Mayorga, C., Cornforth, A. N., and Allen, K. (2009) Phase II trial of dendritic cells loaded with antigens from self-renewing, proliferating autologous tumor cells as patient-specific antitumor vaccines in patients with metastatic melanoma: final report. Cancer biotherapy & radiopharmaceuticals 24, 311-319
51. Chang, J. W., Hsieh, J. J., Shen, Y. C., Ho, E., Chuang, C. K., Chen, Y. R., Liao, S. K., Chen, J. S., Leong, S. P., Hou, M. M., Chang, N. J., and Wang, C. H. (2009) Immunotherapy with dendritic cells pulsed by autologous dactinomycin-induced melanoma apoptotic bodies for patients with malignant melanoma. Melanoma research 19, 309-315
52. Trepiakas, R., Berntsen, A., Hadrup, S. R., Bjorn, J., Geertsen, P. F., Straten, P. T., Andersen, M. H., Pedersen, A. E., Soleimani, A., Lorentzen, T., Johansen, J. S., and Svane, I. M. (2010) Vaccination with autologous dendritic cells pulsed with multiple tumor antigens for treatment of patients with malignant melanoma: results from a phase I/II trial. Cytotherapy 12, 721-734
53. Jacobs, J. F., Punt, C. J., Lesterhuis, W. J., Sutmuller, R. P., Brouwer, H. M., Scharenborg, N. M., Klasen, I. S., Hilbrands, L. B., Figdor, C. G., de Vries, I. J., and Adema, G. J. (2010) Dendritic cell vaccination in combination with anti-CD25 monoclonal antibody treatment: a phase I/II study in metastatic melanoma patients. Clinical cancer research: an official journal of the American Association for Cancer Research 16, 5067-5078
54. Ribas, A., Camacho, L. H., Lee, S. M., Hersh, E. M., Brown, C. K., Richards, J. M., Rodriguez, M. J., Prieto, V. G., Glaspy, J. A., Oseguera, D. K., Hernandez, J., Villanueva, A., Chmielowski, B., Mitsky, P., Bercovici, N., Wasserman, E., Landais, D., and Ross, M. I. (2010) Multicenter phase II study of matured dendritic cells pulsed with melanoma cell line lysates in patients with advanced melanoma. Journal of translational medicine 8, 89
55. Ridolfi, L., Petrini, M., Fiammenghi, L., Granato, A. M., Ancarani, V., Pancisi, E., Scarpi, E., Guidoboni, M., Migliori, G., Sanna, S., Tauceri, F., Verdecchia, G. M., Riccobon, A., Valmorri, L., and Ridolfi, R. (2010) Unexpected high response rate to traditional therapy after dendritic cell-based vaccine in advanced melanoma: update of clinical outcome and subgroup analysis. Clinical & developmental immunology 2010, 504979
56. Cornforth, A. N., Fowler, A. W., Carbonell, D. J., and Dillman, R. O. (2011) Resistance to the proapoptotic effects of interferon-gamma on melanoma cells used in patient-specific dendritic cell immunotherapy is associated with improved overall survival. Cancer immunology, immunotherapy: CII 60, 123-131
57. Lesterhuis, W. J., Schreibelt, G., Scharenborg, N. M., Brouwer, H. M., Gerritsen, M. J., Croockewit, S., Coulie, P. G., Torensma, R., Adema, G. J., Figdor, C. G., de Vries, I. J., and Punt, C. J. (2011) Wild-type and modified gp100 peptide-pulsed dendritic cell vaccination of advanced melanoma patients can lead to long-term clinical responses independent of the peptide used. Cancer immunology, immunotherapy: CII 60, 249-260
58. Bjoern, J., Brimnes, M. K., Andersen, M. H., Thor Straten, P., and Svane, I. M. (2011) Changes in peripheral blood level of regulatory T cells in patients with malignant melanoma during treatment with dendritic cell vaccination and low-dose IL-2. Scandinavian journal of immunology 73, 222-233
59. Steele, J. C., Rao, A., Marsden, J. R., Armstrong, C. J., Berhane, S., Billingham, L. J., Graham, N., Roberts, C., Ryan, G., Uppal, H., Walker, C., Young, L. S., and Steven, N. M. (2011) Phase 1/II trial of a dendritic cell vaccine transfected with DNA encoding melan A and gp100 for patients with metastatic melanoma. Gene therapy 18, 584-593
60. Kim, D. S., Kim, D. H., Goo, B., Cho, Y. H., Park, J. M., Lee, T. H., Kim, H. O., Kim, H. S., Lee, H., Lee, J. D., Byamba, D., Je, J. H., and Lee, M. G. (2011) Immunotherapy of malignant melanoma with tumor lysate-pulsed autologous monocyte-derived dendritic cells. Yonsei medical journal 52, 990-998
61. Ellebaek, E., Engell-Noerregaard, L., Iversen, T. Z., Froesig, T. M., Munir, S., Hadrup, S. R., Andersen, M. H., and Svane, I. M. (2012) Metastatic melanoma patients treated with dendritic cell vaccination, Interleukin-2 and metronomic cyclophosphamide: results from a phase II trial. Cancer immunology, immunotherapy: CII 61, 1791-1804
62. Dillman, R. O., Cornforth, A. N., Depriest, C., McClay, E. F., Amatruda, T. T., de Leon, C., Ellis, R. E., Mayorga, C., Carbonell, D., and Cubellis, J. M. (2012) Tumor stem cell antigens as consolidative active specific immunotherapy: a randomized phase II trial of dendritic cells versus tumor cells in patients with metastatic melanoma. Journal of immunotherapy 35, 641-649
63. Dannull, J., Haley, N. R., Archer, G., Nair, S., Boczkowski, D., Harper, M., De Rosa, N., Pickett, N., Mosca, P. J., Burchette, J., Selim, M. A., Mitchell, D. A., Sampson, J., Tyler, D. S., and Pruitt, S. K. (2013) Melanoma immunotherapy using mature DCs expressing the constitutive proteasome. The Journal of clinical investigation 123, 3135-3145
64. Finkelstein, S. E., Iclozan, C., Bui, M. M., Cotter, M. J., Ramakrishnan, R., Ahmed, J., Noyes, D. R., Cheong, D., Gonzalez, R. J., Heysek, R. V., Berman, C., Lenox, B. C., Janssen, W., Zager, J. S., Sondak, V. K., Letson, G. D., Antonia, S. J., and Gabrilovich, D. I. (2012) Combination of external beam radiotherapy (EBRT) with intratumoral injection of dendritic cells as neo-adjuvant treatment of high-risk soft tissue sarcoma patients. International journal of radiation oncology, biology, physics 82, 924-932
65. Stift, A., Sachet, M., Yagubian, R., Bittermann, C., Dubsky, P., Brostjan, C., Pfragner, R., Niederle, B., Jakesz, R., Gnant, M., and Friedl, J. (2004) Dendritic cell vaccination in medullary thyroid carcinoma. Clinical cancer research: an official journal of the American Association for Cancer Research 10, 2944-2953
66. Kuwabara, K., Nishishita, T., Morishita, M., Oyaizu, N., Yamashita, S., Kanematsu, T., Obara, T., Mimura, Y., Inoue, Y., Kaminishi, M., Kaga, K., Amino, N., Kitaoka, M., Ito, K., Miyauchi, A., Noguchi, S., Uchimaru, K., Akagawa, E., Watanabe, N., Takahashi, T. A., Sato, K., Inazawa, T., Nakaoka, T., and Yamashita, N. (2007) Results of a phase I clinical study using dendritic cell vaccinations for thyroid cancer. Thyroid: official journal of the American Thyroid Association 17, 53-58
67. Bachleitner-Hofmann, T., Friedl, J., Hassler, M., Hayden, H., Dubsky, P., Sachet, M., Rieder, E., Pfragner, R., Brostjan, C., Riss, S., Niederle, B., Gnant, M., and Stift, A. (2009) Pilot trial of autologous dendritic cells loaded with tumor lysate(s) from allogeneic tumor cell lines in patients with metastatic medullary thyroid carcinoma. Oncology reports 21, 1585-1592
68. Yu, J. S., Wheeler, C. J., Zeltzer, P. M., Ying, H., Finger, D. N., Lee, P. K., Yong, W. H., Incardona, F., Thompson, R. C., Riedinger, M. S., Zhang, W., Prins, R. M., and Black, K. L. (2001) Vaccination of malignant glioma patients with peptide-pulsed dendritic cells elicits systemic cytotoxicity and intracranial T-cell infiltration. Cancer research 61, 842-847
69. Yamanaka, R., Abe, T., Yajima, N., Tsuchiya, N., Homma, J., Kobayashi, T., Narita, M., Takahashi, M., and Tanaka, R. (2003) Vaccination of recurrent glioma patients with tumour lysate-pulsed dendritic cells elicits immune responses: results of a clinical phase I/II trial. British journal of cancer 89, 1172-1179
70. Yu, J. S., Liu, G., Ying, H., Yong, W. H., Black, K. L., and Wheeler, C. J. (2004) Vaccination with tumor lysate-pulsed dendritic cells elicits antigen-specific, cytotoxic T-cells in patients with malignant glioma. Cancer research 64, 4973-4979
71. Yamanaka, R., Honma, J., Tsuchiya, N., Yajima, N., Kobayashi, T., and Tanaka, R. (2005) Tumor lysate and IL-18 loaded dendritic cells elicits Th1 response, tumor-specific CD8+ cytotoxic T cells in patients with malignant glioma. Journal of neuro-oncology 72, 107-113
72. Yamanaka, R., Homma, J., Yajima, N., Tsuchiya, N., Sano, M., Kobayashi, T., Yoshida, S., Abe, T., Narita, M., Takahashi, M., and Tanaka, R. (2005) Clinical evaluation of dendritic cell vaccination for patients with recurrent glioma: results of a clinical phase I/II trial. Clinical cancer research: an official journal of the American Association for Cancer Research 11, 4160-4167
73. Liau, L. M., Prins, R. M., Kiertscher, S. M., Odesa, S. K., Kremen, T. J., Giovannone, A. J., Lin, J. W., Chute, D. J., Mischel, P. S., Cloughesy, T. F., and Roth, M. D. (2005) Dendritic cell vaccination in glioblastoma patients induces systemic and intracranial T-cell responses modulated by the local central nervous system tumor microenvironment. Clinical cancer research: an official journal of the American Association for Cancer Research 11, 5515-5525
74. Walker, D. G., Laherty, R., Tomlinson, F. H., Chuah, T., and Schmidt, C. (2008) Results of a phase I dendritic cell vaccine trial for malignant astrocytoma: potential interaction with adjuvant chemotherapy. Journal of clinical neuroscience: official journal of the Neurosurgical Society of Australasia 15, 114-121
75. Leplina, O. Y., Stupak, V. V., Kozlov, Y. P., Pendyurin, I. V., Nikonov, S. D., Tikhonova, M. A., Sycheva, N. V., Ostanin, A. A., and Chernykh, E. R. (2007) Use of interferon-alpha-induced dendritic cells in the therapy of patients with malignant brain gliomas. Bulletin of experimental biology and medicine 143, 528-534
76. De Vleeschouwer, S., Fieuws, S., Rutkowski, S., Van Calenbergh, F., Van Loon, J., Goffin, J., Sciot, R., Wilms, G., Demaerel, P., Warmuth-Metz, M., Soerensen, N., Wolff, J. E., Wagner, S., Kaempgen, E., and Van Gool, S. W. (2008) Postoperative adjuvant dendritic cell-based immunotherapy in patients with relapsed glioblastoma multiforme. Clinical cancer research: an official journal of the American Association for Cancer Research 14, 3098-3104
77. Ardon, H., De Vleeschouwer, S., Van Calenbergh, F., Claes, L., Kramm, C. M., Rutkowski, S., Wolff, J. E., and Van Gool, S. W. (2010) Adjuvant dendritic cell-based tumour vaccination for children with malignant brain tumours. Pediatric blood & cancer 54, 519-525
78. Prins, R. M., Soto, H., Konkankit, V., Odesa, S. K., Eskin, A., Yong, W. H., Nelson, S. F., and Liau, L. M. (2011) Gene expression profile correlates with T-cell infiltration and relative survival in glioblastoma patients vaccinated with dendritic cell immunotherapy. Clinical cancer research: an official journal of the American Association for Cancer Research 17, 1603-1615
79. Okada, H., Kalinski, P., Ueda, R., Hoji, A., Kohanbash, G., Donegan, T. E., Mintz, A. H., Engh, J. A., Bartlett, D. L., Brown, C. K., Zeh, H., Holtzman, M. P., Reinhart, T. A., Whiteside, T. L., Butterfield, L. H., Hamilton, R. L., Potter, D. M., Pollack, I. F., Salazar, A. M., and Lieberman, F. S. (2011) Induction of CD8+ T-cell responses against novel glioma-associated antigen peptides and clinical activity by vaccinations with {alpha}-type 1 polarized dendritic cells and polyinosinic-polycytidylic acid stabilized by lysine and carboxymethylcellulose in patients with recurrent malignant glioma. Journal of clinical oncology: official journal of the American Society of Clinical Oncology 29, 330-336
80. Fadul, C. E., Fisher, J. L., Hampton, T. H., Lallana, E. C., Li, Z., Gui, J., Szczepiorkowski, Z. M., Tosteson, T. D., Rhodes, C. H., Wishart, H. A., Lewis, L. D., and Ernstoff, M. S. (2011) Immune response in patients with newly diagnosed glioblastoma multiforme treated with intranodal autologous tumor lysate-dendritic cell vaccination after radiation chemotherapy. Journal of immunotherapy 34, 382-389
81. Chang, C. N., Huang, Y. C., Yang, D. M., Kikuta, K., Wei, K. J., Kubota, T., and Yang, W. K. (2011) A phase I/II clinical trial investigating the adverse and therapeutic effects of a postoperative autologous dendritic cell tumor vaccine in patients with malignant glioma. Journal of clinical neuroscience: official journal of the Neurosurgical Society of Australasia 18, 1048-1054
82. Cho, D. Y., Yang, W. K., Lee, H. C., Hsu, D. M., Lin, H. L., Lin, S. Z., Chen, C. C., Ham, H. J., Liu, C. L., Lee, W. Y., and Ho, L. H. (2012) Adjuvant immunotherapy with whole-cell lysate dendritic cells vaccine for glioblastoma multiforme: a phase II clinical trial. World neurosurgery 77, 736-744
83. Iwami, K., Shimato, S., Ohno, M., Okada, H., Nakahara, N., Sato, Y., Yoshida, J., Suzuki, S., Nishikawa, H., Shiku, H., Natsume, A., and Wakabayashi, T. (2012) Peptide-pulsed dendritic cell vaccination targeting interleukin-13 receptor alpha2 chain in recurrent malignant glioma patients with HLA-A*24/A*02 allele. Cytotherapy 14, 733-742
84. Fong, B., Jin, R., Wang, X., Safaee, M., Lisiero, D. N., Yang, I., Li, G., Liau, L. M., and Prins, R. M. (2012) Monitoring of regulatory T cell frequencies and expression of CTLA-4 on T cells, before and after DC vaccination, can predict survival in GBM patients. PloS one 7, e32614
85. De Vleeschouwer, S., Ardon, H., Van Calenbergh, F., Sciot, R., Wilms, G., van Loon, J., Goffin, J., and Van Gool, S. (2012) Stratification according to HGG-IMMUNO RPA model predicts outcome in a large group of patients with relapsed malignant glioma treated by adjuvant postoperative dendritic cell vaccination. Cancer immunology, immunotherapy: CII 61, 2105-2112
86. Phuphanich, S., Wheeler, C. J., Rudnick, J. D., Mazer, M., Wang, H., Nuno, M. A., Richardson, J. E., Fan, X., Ji, J., Chu, R. M., Bender, J. G., Hawkins, E. S., Patil, C. G., Black, K. L., and Yu, J. S. (2013) Phase I trial of a multi-epitope-pulsed dendritic cell vaccine for patients with newly diagnosed glioblastoma. Cancer immunology, immunotherapy: CII 62, 125-135
87. Akiyama, Y., Oshita, C., Kume, A., lizuka, A., Miyata, H., Komiyama, M., Ashizawa, T., Yagoto, M., Abe, Y., Mitsuya, K., Watanabe, R., Sugino, T., Yamaguchi, K., and Nakasu, Y. (2012) alpha-type-1 polarized dendritic cell-based vaccination in recurrent high-grade glioma: a phase I clinical trial. BMC cancer 12, 623
88. Prins, R. M., Wang, X., Soto, H., Young, E., Lisiero, D. N., Fong, B., Everson, R., Yong, W. H., Lai, A., Li, G., Cloughesy, T. F., and Liau, L. M. (2013) Comparison of glioma-associated antigen peptide-loaded versus autologous tumor lysate-loaded dendritic cell vaccination in malignant glioma patients. Journal of immunotherapy 36, 152-157
89. Shah, A. H., Bregy, A., Heros, D. O., Komotar, R. J., and Goldberg, J. (2013) Dendritic cell vaccine for recurrent high-grade gliomas in pediatric and adult subjects: clinical trial protocol. Neurosurgery 73, 863-867
90. Reichardt, V. L., Okada, C. Y., Liso, A., Benike, C. J., Stockerl-Goldstein, K. E., Engleman, E. G., Blume, K. G., and Levy, R. (1999) Idiotype vaccination using dendritic cells after autologous peripheral blood stem cell transplantation for multiple myeloma—a feasibility study. Blood 93, 2411-2419
91. Lim, S. H., and Bailey-Wood, R. (1999) Idiotypic protein-pulsed dendritic cell vaccination in multiple myeloma. International journal of cancer. Journal international du cancer 83, 215-222
92. Motta, M. R., Castellani, S., Rizzi, S., Curti, A., Gubinelli, F., Fogli, M., Ferri, E., Cellini, C., Baccarani, M., and Lemoli, R. M. (2003) Generation of dendritic cells from CD14+ monocytes positively selected by immunomagnetic adsorption for multiple myeloma patients enrolled in a clinical trial of anti-idiotype vaccination. British journal of haematology 121, 240-250
93. Reichardt, V. L., Milazzo, C., Brugger, W., Einsele, H., Kanz, L., and Brossart, P. (2003) Idiotype vaccination of multiple myeloma patients using monocyte-derived dendritic cells. Haematologica 88, 1139-1149
94. Guardino, A. E., Rajapaksa, R., Ong, K. H., Sheehan, K., and Levy, R. (2006) Production of myeloid dendritic cells (DC) pulsed with tumor-specific idiotype protein for vaccination of patients with multiple myeloma. Cytotherapy 8, 277-289
95. Lacy, M. Q., Mandrekar, S., Dispenzieri, A., Hayman, S., Kumar, S., Buadi, F., Dingli, D., Litzow, M., Wettstein, P., Padley, D., Kabat, B., Gastineau, D., Rajkumar, S. V., and Gertz, M. A. (2009) Idiotype-pulsed antigen-presenting cells following autologous transplantation for multiple myeloma may be associated with prolonged survival. American journal of hematology 84, 799-802
96. Yi, Q., Szmania, S., Freeman, J., Qian, J., Rosen, N. A., Viswamitra, S., Cottler-Fox, M., Barlogie, B., Tricot, G., and van Rhee, F. (2010) Optimizing dendritic cell-based immunotherapy in multiple myeloma: intranodal injections of idiotype-pulsed CD40 ligand-matured vaccines led to induction of type-1 and cytotoxic T-cell immune responses in patients. British journal of haematology 150, 554-564
97. Rollig, C., Schmidt, C., Bornhauser, M., Ehninger, G., Schmitz, M., and Auffermann-Gretzinger, S. (2011) Induction of cellular immune responses in patients with stage-I multiple myeloma after vaccination with autologous idiotype-pulsed dendritic cells. Journal of immunotherapy 34, 100-106
98. Zahradova, L., Mollova, K., Ocadlikova, D., Kovarova, L., Adam, Z., Krejci, M., Pour, L., Krivanova, A., Sandecka, V., and Hajek, R. (2012) Efficacy and safety of Id-protein-loaded dendritic cell vaccine in patients with multiple myeloma—phase II study results. Neoplasma 59, 440-449
99. Timmerman, J. M., Czerwinski, D. K., Davis, T. A., Hsu, F. J., Benike, C., Hao, Z. M., Taidi, B., Rajapaksa, R., Caspar, C. B., Okada, C. Y., van Beckhoven, A., Liles, T. M., Engleman, E. G., and Levy, R. (2002) Idiotype-pulsed dendritic cell vaccination for B-cell lymphoma: clinical and immune responses in 35 patients. Blood 99, 1517-1526
100. Maier, T., Tun-Kyi, A., Tassis, A., Jungius, K. P., Burg, G., Dummer, R., and Nestle, F. O. (2003) Vaccination of patients with cutaneous T-cell lymphoma using intranodal injection of autologous tumor-lysate-pulsed dendritic cells. Blood 102, 2338-2344
101. Di Nicola, M., Zappasodi, R., Carlo-Stella, C., Mortarini, R., Pupa, S. M., Magni, M., Devizzi, L., Matteucci, P., Baldassari, P., Ravagnani, F., Cabras, A., Anichini, A., and Gianni, A. M. (2009) Vaccination with autologous tumor-loaded dendritic cells induces clinical and immunologic responses in indolent B-cell lymphoma patients with relapsed and measurable disease: a pilot study. Blood 113, 18-27
102. Hus, I., Rolinski, J., Tabarkiewicz, J., Wojas, K., Bojarska-Junak, A., Greiner, J., Giannopoulos, K., Dmoszynska, A., and Schmitt, M. (2005) Allogeneic dendritic cells pulsed with tumor lysates or apoptotic bodies as immunotherapy for patients with early-stage B-cell chronic lymphocytic leukemia. Leukemia 19, 1621-1627
103. Li, L., Giannopoulos, K., Reinhardt, P., Tabarkiewicz, J., Schmitt, A., Greiner, J., Rolinski, J., Hus, I., Dmoszynska, A., Wiesneth, M., and Schmitt, M. (2006) Immunotherapy for patients with acute myeloid leukemia using autologous dendritic cells generated from leukemic blasts. International journal of oncology 28, 855-861
104. Roddie, H., Klammer, M., Thomas, C., Thomson, R., Atkinson, A., Sproul, A., Waterfall, M., Samuel, K., Yin, J., Johnson, P., and Turner, M. (2006) Phase I/II study of vaccination with dendritic-like leukaemia cells for the immunotherapy of acute myeloid leukaemia. British journal of haematology 133, 152-157
105. Litzow, M. R., Dietz, A. B., Bulur, P. A., Butler, G. W., Gastineau, D. A., Hoering, A., Fink, S. R., Letendre, L., Padley, D. J., Paternoster, S. F., Tefferi, A., and Vuk-Pavlovic, S. (2006) Testing the safety of clinical-grade mature autologous myeloid DC in a phase I clinical immunotherapy trial of CML. Cytotherapy 8, 290-298
106. Westermann, J., Kopp, J., van Lessen, A., Hecker, A. C., Baskaynak, G., le Coutre, P., Dohner, K., Dohner, H., Dorken, B., and Pezzutto, A. (2007) Vaccination with autologous non-irradiated dendritic cells in patients with bcr/abl+ chronic myeloid leukaemia. British journal of haematology 137, 297-306
107. Hus, I., Schmitt, M., Tabarkiewicz, J., Radej, S., Wojas, K., Bojarska-Junak, A., Schmitt, A., Giannopoulos, K., Dmoszynska, A., and Rolinski, J. (2008) Vaccination of B-CLL patients with autologous dendritic cells can change the frequency of leukemia antigen-specific CD8+ T cells as well as CD4+CD25+ FoxP3+ regulatory T cells toward an antileukemia response. Leukemia 22, 1007-1017
108. Palma, M., Adamson, L., Hansson, L., Kokhaei, P., Rezvany, R., Mellstedt, H., Osterborg, A., and Choudhury, A. (2008) Development of a dendritic cell-based vaccine for chronic lymphocytic leukemia. Cancer immunology, immunotherapy: CII 57, 1705-1710
109. Van Tendeloo, V. F., Van de Velde, A., Van Driessche, A., Cools, N., Anguille, S., Ladell, K., Gostick, E., Vermeulen, K., Pieters, K., Nijs, G., Stein, B., Smits, E. L., Schroyens, W. A., Gadisseur, A. P., Vrelust, I., Jorens, P. G., Goossens, H., de Vries, I. J., Price, D. A., Oji, Y., Oka, Y., Sugiyama, H., and Berneman, Z. N. (2010) Induction of complete and molecular remissions in acute myeloid leukemia by Wilms' tumor 1 antigen-targeted dendritic cell vaccination. Proc Natl Acad Sci USA 107, 13824-13829
110. Iwashita, Y., Tahara, K., Goto, S., Sasaki, A., Kai, S., Seike, M., Chen, C. L., Kawano, K., and Kitano, S. (2003) A phase I study of autologous dendritic cell-based immunotherapy for patients with unresectable primary liver cancer. Cancer immunology, immunotherapy: CII 52, 155-161
111. Lee, W. C., Wang, H. C., Hung, C. F., Huang, P. F., Lia, C. R., and Chen, M. F. (2005) Vaccination of advanced hepatocellular carcinoma patients with tumor lysate-pulsed dendritic cells: a clinical trial. Journal of immunotherapy 28, 496-504
112. Butterfield, L. H., Ribas, A., Dissette, V. B., Lee, Y., Yang, J. Q., De la Rocha, P., Duran, S. D., Hernandez, J., Seja, E., Potter, D. M., McBride, W. H., Finn, R., Glaspy, J. A., and Economou, J. S. (2006) A phase I/II trial testing immunization of hepatocellular carcinoma patients with dendritic cells pulsed with four alpha-fetoprotein peptides. Clinical cancer research: an official journal of the American Association for Cancer Research 12, 2817-2825
113. Palmer, D. H., Midgley, R. S., Mirza, N., Torr, E. E., Ahmed, F., Steele, J. C., Steven, N. M., Ken, D. J., Young, L. S., and Adams, D. H. (2009) A phase II study of adoptive immunotherapy using dendritic cells pulsed with tumor lysate in patients with hepatocellular carcinoma. Hepatology 49, 124-132
114. El Ansary, M., Mogawer, S., Elhamid, S. A., Alwakil, S., Aboelkasem, F., Sabaawy, H. E., and Abdelhalim, O. (2013) Immunotherapy by autologous dendritic cell vaccine in patients with advanced HCC. Journal of cancer research and clinical oncology 139, 39-48
115. Tada, F., Abe, M., Hirooka, M., Ikeda, Y., Hiasa, Y., Lee, Y., Jung, N. C., Lee, W. B., Lee, H. S., Bae, Y. S., and Onji, M. (2012) Phase I/II study of immunotherapy using tumor antigen-pulsed dendritic cells in patients with hepatocellular carcinoma. International journal of oncology 41, 1601-1609
116. Ueda, Y., Itoh, T., Nukaya, I., Kawashima, I., Okugawa, K., Yano, Y., Yamamoto, Y., Naitoh, K., Shimizu, K., Imura, K., Fuji, N., Fujiwara, H., Ochiai, T., Itoi, H., Sonoyama, T., Hagiwara, A., Takesako, K., and Yamagishi, H. (2004) Dendritic cell-based immunotherapy of cancer with carcinoembryonic antigen-derived, HLA-A24-restricted CTL epitope: Clinical outcomes of 18 patients with metastatic gastrointestinal or lung adenocarcinomas. International journal of oncology 24, 909-917
117. Hirschowitz, E. A., Foody, T., Kryscio, R., Dickson, L., Sturgill, J., and Yannelli, J. (2004) Autologous dendritic cell vaccines for non-small-cell lung cancer. Journal of clinical oncology: official journal of the American Society of Clinical Oncology 22, 2808-2815
118. Chang, G. C., Lan, H. C., Juang, S. H., Wu, Y. C., Lee, H. C., Hung, Y. M., Yang, H. Y., Whang-Peng, J., and Liu, K. J. (2005) A pilot clinical trial of vaccination with dendritic cells pulsed with autologous tumor cells derived from malignant pleural effusion in patients with late-stage lung carcinoma. Cancer 103, 763-771
119. Yannelli, J. R., Sturgill, J., Foody, T., and Hirschowitz, E. (2005) The large scale generation of dendritic cells for the immunization of patients with non-small cell lung cancer (NSCLC). Lung cancer 47, 337-350
120. Ishikawa, A., Motohashi, S., Ishikawa, E., Fuchida, H., Higashino, K., Otsuji, M., lizasa, T., Nakayama, T., Taniguchi, M., and Fujisawa, T. (2005) A phase I study of alpha-galactosylceramide (KRN7000)-pulsed dendritic cells in patients with advanced and recurrent non-small cell lung cancer. Clinical cancer research: an official journal of the American Association for Cancer Research 11, 1910-1917
121. Antonia, S. J., Mirza, N., Fricke, I., Chiappori, A., Thompson, P., Williams, N., Bepler, G., Simon, G., Janssen, W., Lee, J. H., Menander, K., Chada, S., and Gabrilovich, D. I. (2006) Combination of p53 cancer vaccine with chemotherapy in patients with extensive stage small cell lung cancer. Clinical cancer research: an official journal of the American Association for Cancer Research 12, 878-887
122. Perrot, I., Blanchard, D., Freymond, N., Isaac, S., Guibert, B., Pacheco, Y., and Lebecque, S. (2007) Dendritic cells infiltrating human non-small cell lung cancer are blocked at immature stage. Journal of immunology 178, 2763-2769
123. Hirschowitz, E. A., Foody, T., Hidalgo, G. E., and Yannelli, J. R. (2007) Immunization of NSCLC patients with antigen-pulsed immature autologous dendritic cells. Lung cancer 57, 365-372
124. Baratelli, F., Takedatsu, H., Hazra, S., Peebles, K., Luo, J., Kurimoto, P. S., Zeng, G., Batra, R. K., Sharma, S., Dubinett, S. M., and Lee, J. M. (2008) Pre-clinical characterization of GMP grade CCL21-gene modified dendritic cells for application in a phase I trial in non-small cell lung cancer. Journal of translational medicine 6, 38
125. Hegmans, J. P., Veltman, J. D., Lambers, M. E., de Vries, I. J., Figdor, C. G., Hendriks, R. W., Hoogsteden, H. C., Lambrecht, B. N., and Aerts, J. G. (2010) Consolidative dendritic cell-based immunotherapy elicits cytotoxicity against malignant mesothelioma. American journal of respiratory and critical care medicine 181, 1383-1390
126. Um, S. J., Choi, Y. J., Shin, H. J., Son, C. H., Park, Y. S., Roh, M. S., Kim, Y. S., Kim, Y. D., Lee, S. K., Jung, M. H., Lee, M. K., Son, C., Choi, P. J., Chung, J., Kang, C. D., and Lee, E. Y. (2010) Phase I study of autologous dendritic cell tumor vaccine in patients with non-small cell lung cancer. Lung cancer 70, 188-194
127. Chiappori, A. A., Soliman, H., Janssen, W. E., Antonia, S. J., and Gabrilovich, D. I. (2010) INGN-225: a dendritic cell-based p53 vaccine (Ad.p53-DC) in small cell lung cancer: observed association between immune response and enhanced chemotherapy effect. Expert opinion on biological therapy 10, 983-991
128. Perroud, M. W., Jr., Honma, H. N., Barbeiro, A. S., Gilli, S. C., Almeida, M. T., Vassallo, J., Saad, S. T., and Zambon, L. (2011) Mature autologous dendritic cell vaccines in advanced non-small cell lung cancer: a phase I pilot study. Journal of experimental & clinical cancer research: CR 30, 65
129. Skachkova, O. V., Khranovska, N. M., Gorbach, O. I., Svergun, N. M., Sydor, R. I., and Nikulina, V. V. (2013) Immunological markers of anti-tumor dendritic cells vaccine efficiency in patients with non-small cell lung cancer. Experimental oncology 35, 109-113
130. Hernando, J. J., Park, T. W., Kubler, K., Offergeld, R., Schlebusch, H., and Bauknecht, T. (2002) Vaccination with autologous tumour antigen-pulsed dendritic cells in advanced gynaecological malignancies: clinical and immunological evaluation of a phase I trial. Cancer immunology, immunotherapy: CII 51, 45-52
131. Rahma, O. E., Ashtar, E., Czystowska, M., Szajnik, M. E., Wieckowski, E., Bernstein, S., Herrin, V. E., Shams, M. A., Steinberg, S. M., Merino, M., Gooding, W., Visus, C., Deleo, A. B., Wolf, J. K., Bell, J. G., Berzofsky, J. A., Whiteside, T. L., and Khleif, S. N. (2012) A gynecologic oncology group phase II trial of two p53 peptide vaccine approaches: subcutaneous injection and intravenous pulsed dendritic cells in high recurrence risk ovarian cancer patients. Cancer immunology, immunotherapy: CII 61, 373-384
132. Chu, C. S., Boyer, J., Schullery, D. S., Gimotty, P. A., Gamerman, V., Bender, J., Levine, B. L., Coukos, G., Rubin, S. C., Morgan, M. A., Vonderheide, R. H., and June, C. H. (2012) Phase I/II randomized trial of dendritic cell vaccination with or without cyclophosphamide for consolidation therapy of advanced ovarian cancer in first or second remission. Cancer immunology, immunotherapy: CII 61, 629-641
133. Kandalaft, L. E., Chiang, C. L., Tanyi, J., Motz, G., Balint, K., Mick, R., and Coukos, G. (2013) A Phase I vaccine trial using dendritic cells pulsed with autologous oxidized lysate for recurrent ovarian cancer. Journal of translational medicine 11, 149
134. Lepisto, A. J., Moser, A. J., Zeh, H., Lee, K., Bartlett, D., McKolanis, J. R., Geller, B. A., Schmotzer, A., Potter, D. P., Whiteside, T., Finn, O. J., and Ramanathan, R. K. (2008) A phase I/II study of a MUC1 peptide pulsed autologous dendritic cell vaccine as adjuvant therapy in patients with resected pancreatic and biliary tumors. Cancer therapy 6, 955-964
135. Rong, Y., Qin, X., Jin, D., Lou, W., Wu, L., Wang, D., Wu, W., Ni, X., Mao, Z., Kuang, T., Zang, Y. Q., and Qin, X. (2012) A phase I pilot trial of MUC1-peptide-pulsed dendritic cells in the treatment of advanced pancreatic cancer. Clin Exp Med 12, 173-180
136. Endo, H., Saito, T., Kenjo, A., Hoshino, M., Terashima, M., Sato, T., Anazawa, T., Kimura, T., Tsuchiya, T., Irisawa, A., Ohira, H., Hikichi, T., Takagi, T., and Gotoh, M. (2012) Phase I trial of preoperative intratumoral injection of immature dendritic cells and OK-432 for resectable pancreatic cancer patients. Journal of hepato-biliary-pancreatic sciences 19, 465-475
137. Yasukawa, M., Ohminami, H., Kojima, K., Hato, T., Hasegawa, A., Takahashi, T., Hirai, H., and Fujita, S. (2001) HLA class II-restricted antigen presentation of endogenous bcr-abl fusion protein by chronic myelogenous leukemia-derived dendritic cells to CD4(+) T lymphocytes. Blood 98, 1498-1505
138. Dahl, O., Fluge, O., Carlsen, E., Wiig, J. N., Myrvold, H. E., Vonen, B., Podhorny, N., Bjerkeset, O., Eide, T. J., Halvorsen, T. B., Tveit, K. M., and Norwegian Gastrointestinal Cancer, G. (2009) Final results of a randomised phase III study on adjuvant chemotherapy with 5 FU and levamisol in colon and rectum cancer stage II and III by the Norwegian Gastrointestinal Cancer Group. Acta oncologica 48, 368-376
139. O'Connell, M. J., Sargent, D. J., Windschitl, H. E., Shepherd, L., Mahoney, M. R., Krook, J. E., Rayson, S., Morton, R. F., Rowland, K. M., Jr., and Kugler, J. W. (2006) Randomized clinical trial of high-dose levamisole combined with 5-fluorouracil and leucovorin as surgical adjuvant therapy for high-risk colon cancer. Clinical colorectal cancer 6, 133-139
140. Holcombe, R. F., McLaren, C. E., and Milovanovic, T. (2006) Immunomodulation with low dose levamisole in patients with colonic polyps. Cancer detection and prevention 30, 94-98
141. Haller, D. G., Catalano, P. J., Macdonald, J. S., O'Rourke, M. A., Frontiera, M. S., Jackson, D. V., and Mayer, R. J. (2005) Phase III study of fluorouracil, leucovorin, and levamisole in high-risk stage II and III colon cancer: final report of Intergroup 0089. Journal of clinical oncology: official journal of the American Society of Clinical Oncology 23, 8671-8678
142. Tsavaris, N., Gennatas, K., Kosmas, C., Skopelitis, H. M., Gouveris, P., Dimitrakopoulos, A., Zacharakis, M., Kouraklis, G., Vasiliou, J., Felekouras, E., Voros, D., Zografos, G., Balafouta, M., Paraskevaidis, M., Safioleas, M., Fotiadis, K., Papastratis, G., Karatzas, G., and Papalambros, E. (2004) Leucovorin and fluorouracil vs levamisole and fluorouracil as adjuvant chemotherapy in rectal cancer. Oncology reports 12, 927-932
143. Taal, B. G., Van Tinteren, H., Zoetmulder, F. A., and group, N. (2001) Adjuvant 5FU plus levamisole in colonic or rectal cancer: improved survival in stage II and III. British journal of cancer 85, 1437-1443
144. Sinicrope, F. A., Pazdur, R., and Levin, B. (1996) Phase I trial of sulindac plus 5-fluorouracil and levamisole: potential adjuvant therapy for colon carcinoma. Clinical cancer research: an official journal of the American Association for Cancer Research 2, 37-41
145. Dillman, R. O. (2011) Cancer immunotherapy. Cancer biotherapy & radiopharmaceuticals 26, 1-64
146. Sajid, M. S., Iqbal, Z., Muhammad, G., and Iqbal, M. U. (2006) Immunomodulatory effect of various anti-parasitics: a review. Parasitology 132, 301-313
147. Yip, D., Strickland, A. H., Karapetis, C. S., Hawkins, C. A., and Harper, P. G. (2000) Immunomodulation therapy in colorectal carcinoma. Cancer treatment reviews 26, 169-190
148. Chen, L. Y., Lin, Y. L., and Chiang, B. L. (2008) Levamisole enhances immune response by affecting the activation and maturation of human monocyte-derived dendritic cells. Clinical and experimental immunology 151, 174-181
149. Link, K. H., Kornmann, M., Staib, L., Redenbacher, M., Kron, M., Beger, H. G., and Study Group Oncology of Gastrointestinal, T. (2005) Increase of survival benefit in advanced resectable colon cancer by extent of adjuvant treatment: results of a randomized trial comparing modulation of 5-FU+levamisole with folinic acid or with interferon-alpha. Annals of surgery 242, 178-187
150. Schippinger, W., Jagoditsch, M., Sorre, C., Gnant, M., Steger, G., Hausmaninger, H., Mlineritsch, B., Schaberl-Moser, R., Mischinger, H. J., Hofbauer, F., Holzberger, P., Mittlbock, M., Jakesz, R., Austrian, B., and Colorectal Cancer Study, G. (2005) A prospective randomised trial to study the role of levamisole and interferon alfa in an adjuvant therapy with 5-FU for stage III colon cancer. British journal of cancer 92, 1655-1662
151. Magno, C., Melloni, D., Gali, A., Mucciardi, G., Nicocia, G., Morandi, B., Melioli, G., and Ferlazzo, G. (2002) The anti-tumor activity of bacillus Calmette-Guerin in bladder cancer is associated with an increase in the circulating level of interleukin-2. Immunology letters 81, 235-238
152. Lamm, D. L., Thor, D. E., Winters, W. D., Stogdill, V. D., and Radwin, H. M. (1981) BCG immunotherapy of bladder cancer: inhibition of tumor recurrence and associated immune responses. Cancer 48, 82-88
153. O'Connell, M. J., Ritts, R. E., Jr., Moertel, C. G., O'Fallon, J. R., Rubin, J., Frytak, S., and Schutt, A. J. (1981) A controlled clinical and immunological evaluation of immunotherapy with Bacillus Calmette—Guerin in patients with metastatic cancer. Cancer clinical trials 4, 439-449
154. Haghbin, M., Cunningham-Rundles, S., Thaler, H. T., Gupta, S., Hecht, S., Murphy, M. L., and Oettgen, H. F. (1980) Immunotherapy with oral BCG and serial immune evaluation in childhood lymphoblastic leukemia following three years of chemotherapy. Cancer 46, 2577-2586
155. Guinan, P., Crispen, R., Baumgartner, G., Rao, R., Totonchi, E., Ablin, R., and John, T. (1979) Adjuvant immunotherapy with bacillus Calmette-Guerin in prostatic cancer. Urology 14, 561-565
156. O'Connell, M. J., Moertel, C. G., Ritts, R. E., Jr., Frytak, S., and Reitemeier, R. J. (1979) A comparative clinical and immunological assessment of methanol extraction residue of Bacillus Calmette-Guerin versus placebo in patients with advanced cancer. Cancer research 39, 3720-3724
157. Olkowski, Z. L., McLaren, J. R., and Skeen, M. J. (1978) Effects of combined immunotherapy with levamisole and Bacillus Calmette-Guerin on immunocompetence of patients with squamous cell carcinoma of the cervix, head and neck, and lung undergoing radiation therapy. Cancer treatment reports 62, 1651-1661
158. Cohen, M. H., Jessup, J. M., Felix, E. L., Weese, J. L., and Herberman, R. B. (1978) Intralesional treatment of recurrent metastatic cutaneous malignant melanoma: a randomized prospective study of intralesional Bacillus Calmette-Guerin versus intralesional dinitrochlorobenzene. Cancer 41, 2456-2463
159. Robinson, E., Bartal, A., Cohen, Y., Haasz, R., and Mekori, T. (1977) Treatment of lung cancer by radiotherapy, chemotherapy, and methanol extraction residue of BCG (MER): clinical and immunological studies. Cancer 40, 1052-1059
160. Sparks, F. C., Wile, A. G., Ramming, K. P., Silver, H. K., Wolk, R. W., and Morton, D. L. (1976) Immunology and adjuvant chemoimmunotherapy of breast cancer. Archives of surgery 111, 1057-1062
161. Sharma, P., Bajorin, D. F., Jungbluth, A. A., Herr, H., Old, L. J., and Gnjatic, S. (2008) Immune responses detected in urothelial carcinoma patients after vaccination with NY-ESO-1 protein plus BCG and GM-CSF. Journal of immunotherapy 31, 849-857
162. Berger, M., Kreutz, F. T., Horst, J. L., Baldi, A. C., and Koff, W. J. (2007) Phase I study with an autologous tumor cell vaccine for locally advanced or metastatic prostate cancer. Journal of pharmacy & pharmaceutical sciences: a publication of the Canadian Society for Pharmaceutical Sciences, Societe canadienne des sciences pharmaceutiques 10, 144-152
163. Barrio, M. M., de Motta, P. T., Kaplan, J., von Euw, E. M., Bravo, A. I., Chacon, R. D., and Mordoh, J. (2006) A phase I study of an allogeneic cell vaccine (VACCIMEL) with GM-CSF in melanoma patients. Journal of immunotherapy 29, 444-454
164. Raez, L. E., Fein, S., and Podack, E. R. (2005) Lung cancer immunotherapy. Clinical medicine & research 3, 221-228
165. Hsueh, E. C., Essner, R., Foshag, L. J., Ye, W., and Morton, D. L. (2002) Active immunotherapy by reinduction with a polyvalent allogeneic cell vaccine correlates with improved survival in recurrent metastatic melanoma. Annals of surgical oncology 9, 486-492
166. Tsioulias, G. J., Gupta, R. K., Tisman, G., Hsueh, E. C., Essner, R., Wanek, L. A., and Morton, D. L. (2001) Serum TA90 antigen-antibody complex as a surrogate marker for the efficacy of a polyvalent allogeneic whole-cell vaccine (CancerVax) in melanoma. Annals of surgical oncology 8, 198-203
167. Li, Q., Normolle, D. P., Sayre, D. M., Zeng, X., Sun, R., Jiang, G., Redman, B. D., and Chang, A. E. (2000) Immunological effects of BCG as an adjuvant in autologous tumor vaccines. Clinical immunology 94, 64-72
168. Diederichsen, A. C., Stenholm, A. C., Kronborg, O., Fenger, C., Jensenius, J. C., Zeuthen, J., Kristensen, T., and Christensen, P. B. (1998) Immunisation of colorectal cancer patients with autologous tumour cells. Oncology reports 5, 823-826
169. Saha, K., Kapila, H., Madan, R., and Shinghal, R. N. (1986) Immunologic tools to decipher efficacy of BCG immunotherapy in advanced breast cancer: a one year follow up study. Asian Pacific journal of allergy and immunology/launched by the Allergy and Immunology Society of Thailand 4, 139-148
170. linuma, H., Fukushima, R., Inaba, T., Tamura, J., Inoue, T., Ogawa, E., Horikawa, M., Ikeda, Y., Matsutani, N., Takeda, K., Yoshida, K., Tsunoda, T., Ikeda, T., Nakamura, Y., and Okinaga, K. (2014) Phase I clinical study of multiple epitope peptide vaccine combined with chemoradiation therapy in esophageal cancer patients. Journal of translational medicine 12, 84
171. Hazama, S., Nakamura, Y., Takenouchi, H., Suzuki, N., Tsunedomi, R., Inoue, Y., Tokuhisa, Y., lizuka, N., Yoshino, S., Takeda, K., Shinozaki, H., Kamiya, A., Furukawa, H., and Oka, M. (2014) A phase I study of combination vaccine treatment of five therapeutic epitope-peptides for metastatic colorectal cancer; safety, immunological response, and clinical outcome. Journal of translational medicine 12, 63
172. Salerno, E. P., Shea, S. M., Olson, W. C., Petroni, G. R., Smolkin, M. E., McSkimming, C., Chianese-Bullock, K. A., and Slingluff, C. L., Jr. (2013) Activation, dysfunction and retention of T cells in vaccine sites after injection of incomplete Freund's adjuvant, with or without peptide. Cancer immunology, immunotherapy: CII 62, 1149-1159
173. Slingluff, C. L., Jr., Lee, S., Zhao, F., Chianese-Bullock, K. A., Olson, W. C., Butterfield, L. H., Whiteside, T. L., Leming, P. D., and Kirkwood, J. M. (2013) A randomized phase II trial of multiepitope vaccination with melanoma peptides for cytotoxic T cells and helper T cells for patients with metastatic melanoma (E1602). Clinical cancer research: an official journal of the American Association for Cancer Research 19, 4228-4238
174. Ishikawa, H., Imano, M., Shiraishi, O., Yasuda, A., Peng, Y. F., Shinkai, M., Yasuda, T., Imamoto, H., and Shiozaki, H. (2014) Phase I clinical trial of vaccination with LY6K-derived peptide in patients with advanced gastric cancer. Gastric cancer: official journal of the International Gastric Cancer Association and the Japanese Gastric Cancer Association 17, 173-180
175. Kawaguchi, S., Tsukahara, T., Ida, K., Kimura, S., Murase, M., Kano, M., Emori, M., Nagoya, S., Kaya, M., Torigoe, T., Ueda, E., Takahashi, A., Ishii, T., Tatezaki, S., Toguchida, J., Tsuchiya, H., Osanai, T., Sugita, T., Sugiura, H., leguchi, M., Ihara, K., Hamada, K., Kakizaki, H., Morii, T., Yasuda, T., Tanizawa, T., Ogose, A., Yabe, H., Yamashita, T., Sato, N., and Wada, T. (2012) SYT-SSX breakpoint peptide vaccines in patients with synovial sarcoma: a study from the Japanese Musculoskeletal Oncology Group. Cancer science 103, 1625-1630
176. Harris, R. C., Chianese-Bullock, K. A., Petroni, G. R., Schaefer, J. T., Brill, L. B., 2nd, Molhoek, K. R., Deacon, D. H., Patterson, J. W., and Slingluff, C. L., Jr. (2012) The vaccine-site microenvironment induced by injection of incomplete Freund's adjuvant, with or without melanoma peptides. Journal of immunotherapy 35, 78-88
177. Schwartzentruber, D. J., Lawson, D. H., Richards, J. M., Conry, R. M., Miller, D. M., Treisman, J., Gailani, F., Riley, L., Conlon, K., Pockaj, B., Kendra, K. L., White, R. L., Gonzalez, R., Kuzel, T. M., Curti, B., Leming, P. D., Whitman, E. D., Balkissoon, J., Reintgen, D. S., Kaufman, H., Marincola, F. M., Merino, M. J., Rosenberg, S. A., Choyke, P., Vena, D., and Hwu, P. (2011) gp100 peptide vaccine and interleukin-2 in patients with advanced melanoma. The New England journal of medicine 364, 2119-2127
178. Rosenberg, S. A., Yang, J. C., Kammula, U. S., Hughes, M. S., Restifo, N. P., Schwarz, S. L., Morton, K. E., Laurencot, C. M., and Sherry, R. M. (2010) Different adjuvanticity of incomplete freund's adjuvant derived from beef or vegetable components in melanoma patients immunized with a peptide vaccine. Journal of immunotherapy 33, 626-629
179. Kameshima, H., Tsuruma, T., Kutomi, G., Shima, H., Iwayama, Y., Kimura, Y., Imamura, M., Torigoe, T., Takahashi, A., Hirohashi, Y., Tamura, Y., Tsukahara, T., Kanaseki, T., Sato, N., and Hirata, K. (2013) Immunotherapeutic benefit of alpha-interferon (IFNalpha) in survivin2B-derived peptide vaccination for advanced pancreatic cancer patients. Cancer science 104, 124-129
180. Sun, C., Dobi, A., Mohamed, A., Li, H., Thangapazham, R. L., Furusato, B., Shaheduzzaman, S., Tan, S. H., Vaidyanathan, G., Whitman, E., Hawksworth, D. J., Chen, Y., Nau, M., Patel, V., Vahey, M., Gutkind, J. S., Sreenath, T., Petrovics, G., Sesterhenn, I. A., McLeod, D. G., and Srivastava, S. (2008) TMPRSS2-ERG fusion, a common genomic alteration in prostate cancer activates C-MYC and abrogates prostate epithelial differentiation. Oncogene 27, 5348-5353
181. Saramaki, O. R., Harjula, A. E., Martikainen, P. M., Vessella, R. L., Tammela, T. L., and Visakorpi, T. (2008) TMPRSS2:ERG fusion identifies a subgroup of prostate cancers with a favorable prognosis. Clin Cancer Res 14, 3395-3400
182. Attard, G., Swennenhuis, J. F., Olmos, D., Reid, A. H., Vickers, E., A′Hern, R., Levink, R., Coumans, F., Moreira, J., Riisnaes, R., Oommen, N. B., Hawche, G., Jameson, C., Thompson, E., Sipkema, R., Carden, C. P., Parker, C., Dearnaley, D., Kaye, S. B., Cooper, C. S., Molina, A., Cox, M. E., Terstappen, L. W., and de Bono, J. S. (2009) Characterization of ERG, AR and PTEN gene status in circulating tumor cells from patients with castration-resistant prostate cancer. Cancer Res 69, 2912-2918
183. Krohn, A., Diedler, T., Burkhardt, L., Mayer, P. S., De Silva, C., Meyer-Kornblum, M., Kotschau, D., Tennstedt, P., Huang, J., Gerhauser, C., Mader, M., Kurtz, S., Sirma, H., Saad, F., Steuber, T., Graefen, M., Plass, C., Sauter, G., Simon, R., Minner, S., and Schlomm, T. (2012) Genomic deletion of PTEN is associated with tumor progression and early PSA recurrence in ERG fusion-positive and fusion-negative prostate cancer. Am J Pathol 181, 401-412
184. Kron, K., Liu, L., Trudel, D., Pethe, V., Trachtenberg, J., Fleshner, N., Bapat, B., and van der Kwast, T. (2012) Correlation of ERG expression and DNA methylation biomarkers with adverse clinicopathologic features of prostate cancer. Clin Cancer Res 18, 2896-2904
185. Grupp, K., Ospina-Klinck, D., Tsourlakis, M. C., Koop, C., Wilczak, W., Adam, M., Simon, R., Sauter, G., Izbicki, J. R., Graefen, M., Huland, H., Steurer, S., Schlomm, T., Minner, S., and Quaas, A. (2014) NY-ESO-1 expression is tightly linked to TMPRSS2-ERG fusion in prostate cancer. Prostate 74, 1012-1022
186. Gsponer, J. R., Braun, M., Scheble, V. J., Zellweger, T., Bachmann, A., Perner, S., Vlajnic, T., Srivastava, M., Tan, S. H., Dobi, A., Sesterhenn, I. A., Srivastava, S., Bubendorf, L., and Ruiz, C. (2014) ERG rearrangement and protein expression in the progression to castration-resistant prostate cancer. Prostate Cancer Prostatic Dis 17, 126-131
187. Font-Tello, A., Juanpere, N., de Muga, S., Lorenzo, M., Lorente, J. A., Fumado, L., Serrano, L., Serrano, S., Lloreta, J., and Hernandez, S. (2015) Association of ERG and TMPRSS2-ERG with grade, stage, and prognosis of prostate cancer is dependent on their expression levels. Prostate 75, 1216-1226
188. Kulda, V., Topolcan, O., Kucera, R., Kripnerova, M., Srbecka, K., Hora, M., Hes, O., Klecka, J., Babuska, V., Rousarova, M., Benson, V., and Pesta, M. (2016) Prognostic Significance of TMPRSS2-ERG Fusion Gene in Prostate Cancer. Anticancer Res 36, 4787-4793
189. Xiao, L., Lanz, R. B., Frolov, A., Castro, P. D., Zhang, Z., Dong, B., Xue, W., Jung, S. Y., Lydon, J. P., Edwards, D. P., Mancini, M. A., Feng, Q., Ittmann, M. M., and He, B. (2016) The Germ Cell Gene TDRD1 as an ERG Target Gene and a Novel Prostate Cancer Biomarker. Prostate 76, 1271-1284
190. Reig, O., Marin-Aguilera, M., Carrera, G., Jimenez, N., Pare, L., Garcia-Recio, S., Gaba, L., Pereira, M. V., Fernandez, P., Prat, A., and Mellado, B. (2016) TMPRSS2-ERG in Blood and Docetaxel Resistance in Metastatic Castration-resistant Prostate Cancer. Eur Urol 70, 709-713
191. Gaiger, A., Reese, V., Disis, M. L., and Cheever, M. A. (2000) Immunity to WT1 in the animal model and in patients with acute myeloid leukemia. Blood 96, 1480-1489
192. Gaiger, A., Carter, L., Greinix, H., Carter, D., McNeill, P. D., Houghton, R. L., Cornellison, C. D., Vedvick, T. S., Skeiky, Y. A., and Cheever, M. A. (2001) WT1-specific serum antibodies in patients with leukemia. Clin Cancer Res 7, 761s-765s 193. Elisseeva, O. A., Oka, Y., Tsuboi, A., Ogata, K., Wu, F., Kim, E. H., Soma, T., Tamaki, H., Kawakami, M., Oji, Y., Hosen, N., Kubota, T., Nakagawa, M., Yamagami, T., Hiraoka, A., Tsukaguchi, M., Udaka, K., Ogawa, H., Kishimoto, T., Nomura, T., and Sugiyama, H. (2002) Humoral immune responses against Wilms tumor gene WT1 product in patients with hematopoietic malignancies. Blood 99, 3272-3279
194. Rosenfeld, C., Cheever, M. A., and Gaiger, A. (2003) WT1 in acute leukemia, chronic myelogenous leukemia and myelodysplastic syndrome: therapeutic potential of WT1 targeted therapies. Leukemia 17, 1301-1312
195. Nakajima, H., Kawasaki, K., Oka, Y., Tsuboi, A., Kawakami, M., Ikegame, K., Hoshida, Y., Fujiki, F., Nakano, A., Masuda, T., Wu, F., Taniguchi, Y., Yoshihara, S., Elisseeva, O. A., Oji, Y., Ogawa, H., Azuma, I., Kawase, I., Aozasa, K., and Sugiyama, H. (2004) WT1 peptide vaccination combined with BCG-CWS is more efficient for tumor eradication than WT1 peptide vaccination alone. Cancer Immunol Immunother 53, 617-624
196. Wu, F., Oka, Y., Tsuboi, A., Elisseeva, O. A., Ogata, K., Nakajima, H., Fujiki, F., Masuda, T., Murakami, M., Yoshihara, S., Ikegame, K., Hosen, N., Kawakami, M., Nakagawa, M., Kubota, T., Soma, T., Yamagami, T., Tsukaguchi, M., Ogawa, H., Oji, Y., Hamaoka, T., Kawase, I., and Sugiyama, H. (2005) Th1-biased humoral immune responses against Wilms tumor gene WT1 product in the patients with hematopoietic malignancies. Leukemia 19, 268-274
197. Guo, Y., Niiya, H., Azuma, T., Uchida, N., Yakushijin, Y., Sakai, I., Hato, T., Takahashi, M., Senju, S., Nishimura, Y., and Yasukawa, M. (2005) Direct recognition and lysis of leukemia cells by WT1-specific CD4+ T lymphocytes in an HLA class II-restricted manner. Blood 106, 1415-1418
198. Gannage, M., Abel, M., Michallet, A. S., Delluc, S., Lambert, M., Giraudier, S., Kratzer, R., Niedermann, G., Saveanu, L., Guilhot, F., Camoin, L., Varet, B., Buzyn, A., and Caillat-Zucman, S. (2005) Ex vivo characterization of multiepitopic tumor-specific CD8 T cells in patients with chronic myeloid leukemia: implications for vaccine development and adoptive cellular immunotherapy. J Immunol 174, 8210-8218
199. Lee, H. M., Oh, B. C., Yang, J. H., Cho, J., Lee, G., Lee, D. S., Hwang, W. S., and Lee, J. R. (2006) Age-dependent expression of immune-privilege and proliferation-related molecules on porcine Sertoli cells. Xenotransplantation 13, 69-74
200. Pinilla-Ibarz, J., May, R. J., Korontsvit, T., Gomez, M., Kappel, B., Zakhaleva, V., Zhang, R. H., and Scheinberg, D. A. (2006) Improved human T-cell responses against synthetic HLA-0201 analog peptides derived from the WT1 oncoprotein. Leukemia 20, 2025-2033
201. Oka, Y., Tsuboi, A., Elisseeva, O. A., Nakajima, H., Fujiki, F., Kawakami, M., Shirakata, T., Nishida, S., Hosen, N., Oji, Y., Kawase, I., and Sugiyama, H. (2007) WT1 peptide cancer vaccine for patients with hematopoietic malignancies and solid cancers. ScientificWorldJoumal 7, 649-665
202. Oji, Y., Kitamura, Y., Kamino, E., Kitano, A., Sawabata, N., Inoue, M., Mori, M., Nakatsuka, S., Sakaguchi, N., Miyazaki, K., Nakamura, M., Fukuda, I., Nakamura, J., Tatsumi, N., Takakuwa, T., Nishida, S., Shirakata, T., Hosen, N., Tsuboi, A., Nezu, R., Maeda, H., Oka, Y., Kawase, I., Aozasa, K., Okumura, M., Miyoshi, S., and Sugiyama, H. (2009) WT1 IgG antibody for early detection of nonsmall cell lung cancer and as its prognostic factor. Int J Cancer 125, 381-387
203. Saitoh, A., Narita, M., Watanabe, N., Tochiki, N., Yamahira, A., Nakamura, T., Kaji, M., Masuko, M., Furukawa, T., Toba, K., Fuse, I., Aizawa, Y., and Takahashi, M. (2011) WT1 peptide vaccination in a CML patient: induction of effective cytotoxic T lymphocytes and significance of peptide administration interval. Med Oncol 28, 219-230
204. Maslak, P. G., Dao, T., Krug, L. M., Chanel, S., Korontsvit, T., Zakhaleva, V., Zhang, R., Wolchok, J. D., Yuan, J., Pinilla-Ibarz, J., Berman, E., Weiss, M., Jurcic, J., Frattini, M. G., and Scheinberg, D. A. (2010) Vaccination with synthetic analog peptides derived from WT1 oncoprotein induces T-cell responses in patients with complete remission from acute myeloid leukemia. Blood 116, 171-179
205. Oji, Y., Oka, Y., Nishida, S., Tsuboi, A., Kawakami, M., Shirakata, T., Takahashi, K., Murao, A., Nakajima, H., Narita, M., Takahashi, M., Morita, S., Sakamoto, J., Tanaka, T., Kawase, I., Hosen, N., and Sugiyama, H. (2010) WT1 peptide vaccine induces reduction in minimal residual disease in an Imatinib-treated CML patient. Eur J Haematol 85, 358-360
206. Morimoto, S., Oka, Y., Tsuboi, A., Tanaka, Y., Fujiki, F., Nakajima, H., Hosen, N., Nishida, S., Nakata, J., Nakae, Y., Maruno, M., Myoui, A., Enomoto, T., Izumoto, S., Sekimoto, M., Kagawa, N., Hashimoto, N., Yoshimine, T., Oji, Y., Kumanogoh, A., and Sugiyama, H. (2012) Biased usage of T cell receptor beta-chain variable region genes of Wilms' tumor gene (WT1)-specific CD8+ T cells in patients with solid tumors and healthy donors. Cancer Sci 103, 408-414
207. Babiarova, K., Kutinova, L., Zurkova, K., Krystofova, J., Brabcova, E., Hainz, P., Musil, J., and Nemeckova, S. (2012) Immunization with WT1-derived peptides by tattooing inhibits the growth of TRAMP-C2 prostate tumor in mice. J Immunother 35, 478-487
208. Rein, L. A., and Chao, N. J. (2014) WT1 vaccination in acute myeloid leukemia: new methods of implementing adoptive immunotherapy. Expert Opin Investig Drugs 23, 417-426
209. Tan, Z., Zhou, J., Cheung, A. K., Yu, Z., Cheung, K. W., Liang, J., Wang, H., Lee, B. K., Man, K., Liu, L., Yuen, K. Y., and Chen, Z. (2014) Vaccine-elicited CD8+ T cells cure mesothelioma by overcoming tumor-induced immunosuppressive environment. Cancer Res 74, 6010-6021
210. Lindstedt, I., Lindgren, M. A., Andersson, E., and Engstrom, W. (2014) The WT1 gene—its role in tumourigenesis and prospects for immunotherapeutic advances. In Vivo 28, 675-681
211. Massaoka, M. H., Matsuo, A. L., Figueiredo, C. R., Girola, N., Farias, C. F., Pasqualoto, K., and Travassos, L. R. (2014) A subtraction tolerization method of immunization allowed for Wilms' tumor protein-1 (WT1) identification in melanoma and discovery of an antitumor peptide sequence. J Immunol Methods 414, 11-19
212. Lutz, M., Worschech, A., Alb, M., Gahn, S., Bernhard, L., Schwab, M., Obermeier, S., Einsele, H., Kammerer, U., Heuschmann, P., Klinker, E., Otto, C., and Mielke, S. (2015) Boost and loss of immune responses against tumor-associated antigens in the course of pregnancy as a model for allogeneic immunotherapy. Blood 125, 261-272
213. Saito, S., Yanagisawa, R., Yoshikawa, K., Higuchi, Y., Koya, T., Yoshizawa, K., Tanaka, M., Sakashita, K., Kobayashi, T., Kurata, T., Hirabayashi, K., Nakazawa, Y., Shiohara, M., Yonemitsu, Y., Okamoto, M., Sugiyama, H., Koike, K., and Shimodaira, S. (2015) Safety and tolerability of allogeneic dendritic cell vaccination with induction of Wilms tumor 1-specific T cells in a pediatric donor and pediatric patient with relapsed leukemia: a case report and review of the literature. Cytotherapy 17, 330-335
214. Wagner, K. D., Cherfils-Vicini, J., Hosen, N., Hohenstein, P., Gilson, E., Hastie, N. D., Michiels, J. F., and Wagner, N. (2014) The Wilms' tumour suppressor WO is a major regulator of tumour angiogenesis and progression. Nat Commun 5, 5852
215. Mayanagi, S., Kitago, M., Sakurai, T., Matsuda, T., Fujita, T., Higuchi, H., Taguchi, J., Takeuchi, H., Itano, O., Aiura, K., Hamamoto, Y., Takaishi, H., Okamoto, M., Sunamura, M., Kawakami, Y., and Kitagawa, Y. (2015) Phase I pilot study of Wilms tumor gene 1 peptide-pulsed dendritic cell vaccination combined with gemcitabine in pancreatic cancer. Cancer Sci 106, 397-406
216. Brayer, J., Lancet, J. E., Powers, J., List, A., Balducci, L., Komrokji, R., and Pinilla-Ibarz, J. (2015) WT1 vaccination in AML and MDS: A pilot trial with synthetic analog peptides. Am J Hematol 90, 602-607
217. Casalegno-Garduno, R., Schmitt, A., Spitschak, A., Greiner, J., Wang, L., Hilgendorf, I., Hirt, C., Ho, A. D., Freund, M., and Schmitt, M. (2016) Immune responses to WT1 in patients with AML or MDS after chemotherapy and allogeneic stem cell transplantation. Int J Cancer 138, 1792-1801
218. Shimodaira, S., Sano, K., Hirabayashi, K., Koya, T., Higuchi, Y., Mizuno, Y., Yamaoka, N., Yuzawa, M., Kobayashi, T., Ito, K., and Koizumi, T. (2015) Dendritic Cell-Based Adjuvant Vaccination Targeting Wilms' Tumor 1 in Patients with Advanced Colorectal Cancer. Vaccines (Basel) 3, 1004-1018
219. Smits, E. L., Stein, B., Nijs, G., Lion, E., Van Tendeloo, V. F., Willemen, Y., Anguille, S., and Berneman, Z. N. (2016) Generation and Cryopreservation of Clinical Grade Wilms' Tumor 1 mRNA-Loaded Dendritic Cell Vaccines for Cancer Immunotherapy. Methods Mol Biol 1393, 27-35
220. Ottensmeier, C., Bowers, M., Hamid, D., Maishman, T., Regan, S., Wood, W., Cazaly, A., and Stanton, L. (2016). in Wilms' tumour antigen 1 Immunity via DNA fusion gene vaccination in haematological malignancies by intramuscular injection followed by intramuscular electroporation: a Phase II non-randomised clinical trial (WIN), Southampton (UK). pp
221. Oji, Y., Hashimoto, N., Tsuboi, A., Murakami, Y., Iwai, M., Kagawa, N., Chiba, Y., Izumoto, S., Elisseeva, O., Ichinohasama, R., Sakamoto, J., Morita, S., Nakajima, H., Takashima, S., Nakae, Y., Nakata, J., Kawakami, M., Nishida, S., Hosen, N., Fujiki, F., Morimoto, S., Adachi, M., Iwamoto, M., Oka, Y., Yoshimine, T., and Sugiyama, H. (2016) Association of WT1 IgG antibody against WT1 peptide with prolonged survival in glioblastoma multiforme patients vaccinated with WT1 peptide. Int J Cancer 139, 1391-1401
222. Benteyn, D., Heirman, C., Thielemans, K., and Bonehill, A. (2016) Engineering WT1-Encoding mRNA to Increase Translational Efficiency in Dendritic Cells. Methods Mol Biol 1428, 115-123
223. Coosemans, A., Tuyaerts, S., Morias, K., Corthals, J., Heirman, C., Thielemans, K., Van Gool, S. W., Vergote, I., and Amant, F. (2016) mRNA Electroporation of Dendritic Cells with WT1, Survivin, and TriMix (a Mixture of caTLR4, CD40L, and CD70). Methods Mol Biol 1428, 277-283
224. Naitoh, K., Kamigaki, T., Matsuda, E., Ibe, H., Okada, S., Oguma, E., Kinoshita, Y., Takimoto, R., Makita, K., Ogasawara, S., and Goto, S. (2016) Immunohistochemical Analysis of WT1 Antigen Expression in Various Solid Cancer Cells. Anticancer Res 36, 3715-3724
225. Nishida, S., and Sugiyama, H. (2016) Immunotherapy Targeting WT1: Designing a Protocol for WT1 Peptide-Based Cancer Vaccine. Methods Mol Biol 1467, 221-232
226. Cebinelli, G. C., N, D. E. S. P., Sena, M. M., CE, D. E. O., Fujita, T. C., SP, D. A. R., FJ, D. E. A. O., Marinello, P. C., and Watanabe, M. A. (2016) Immunotherapy in Acute Leukemias: Implications and Perspectives Using WO Antigen. Anticancer Res 36, 3795-3802
227. Fromm, P. D., Papadimitrious, M. S., Hsu, J. L., Van Kooten Losio, N., Verma, N. D., Lo, T. H., Silveira, P. A., Bryant, C. E., Turtle, C. J., Prue, R. L., Vukovic, P., Munster, D. J., Nagasaki, T., Barnard, R. T., Mahler, S. M., Anguille, S. A., Berneman, Z., Horvath, L. G., Bradstock, K. F., Joshua, D. E., Clark, G. J., and Hart, D. N. (2016) CMRF-56(+) blood dendritic cells loaded with mRNA induce effective antigen-specific cytotoxic T-lymphocyte responses. Oncoimmunology 5, el168555
228. Shimodaira, S., Hirabayashi, K., Yanagisawa, R., Higuchi, Y., Sano, K., and Koizumi, T. (2016) Dendritic Cell-Based Cancer Immunotherapy Targeting Wilms' Tumor 1 for Pediatric Cancer. in Wilms Tumor (van den Heuvel-Eibrink, M. M. ed.), Brisbane (AU). pp
229. Shah, N. N., Loeb, D. M., Khuu, H., Stroncek, D., Ariyo, T., Raffeld, M., Delbrook, C., Mackall, C. L., Wayne, A. S., and Fry, T. J. (2016) Induction of Immune Response after Allogeneic Wilms' Tumor 1 Dendritic Cell Vaccination and Donor Lymphocyte Infusion in Patients with Hematologic Malignancies and Post-Transplantation Relapse. Biol Blood Marrow Transplant 22, 2149-2154
230. Dagvadorj, N., Deuretzbacher, A., Weisenberger, D., Baumeister, E., Trebing, J., Lang, I., Kochel, C., Kapp, M., Kapp, K., Beilhack, A., Hunig, T., Einsele, H., Wajant, H., and Grigoleit, G. U. (2017) Targeting of the WT191-138 fragment to human dendritic cells improves leukemia-specific T-cell responses providing an alternative approach to WT1-based vaccination. Cancer Immunol Immunother 66, 319-332
231. Koido, S., Okamoto, M., Shimodaira, S., and Sugiyama, H. (2016) Wilms' tumor 1 (WT1)-targeted cancer vaccines to extend survival for patients with pancreatic cancer. Immunotherapy 8, 1309-1320
232. Hughes, A., Clarson, J., Tang, C., Vidovic, L., White, D. L., Hughes, T. P., and Yong, A. S. (2017) CML patients with deep molecular responses to TKI have restored immune effectors and decreased PD-1 and immune suppressors. Blood 129, 1166-1176
233. Walters, J. N., Ferraro, B., Duperret, E. K., Kraynyak, K. A., Chu, J., Saint-Fleur, A., Yan, J., Levitsky, H., Khan, A. S., Sardesai, N. Y., and Weiner, D. B. (2017) A Novel DNA Vaccine Platform Enhances Neo-antigen-like T Cell Responses against WT1 to Break Tolerance and Induce Anti-tumor Immunity. Mol Ther 25, 976-988
234. Maturu, P., Jones, D., Ruteshouser, E. C., Hu, Q., Reynolds, J. M., Hicks, J., Putluri, N., Ekmekcioglu, S., Grimm, E. A., Dong, C., and Overwijk, W. W. (2017) Role of Cyclooxygenase-2 Pathway in Creating an Immunosuppressive Microenvironment and in Initiation and Progression of Wilms' Tumor. Neoplasia 19, 237-249
235. Fukuda, K., Funakoshi, T., Sakurai, T., Nakamura, Y., Mori, M., Tanese, K., Tanikawa, A., Taguchi, J., Fujita, T., Okamoto, M., Amagai, M., and Kawakami, Y. (2017) Peptide-pulsed dendritic cell vaccine in combination with carboplatin and paclitaxel chemotherapy for stage IV melanoma. Melanoma Res 27, 326-334
236. Oji, Y., Inoue, M., Takeda, Y., Hosen, N., Shintani, Y., Kawakami, M., Harada, T., Murakami, Y., Iwai, M., Fukuda, M., Nishida, S., Nakata, J., Nakae, Y., Takashima, S., Shirakata, T., Nakajima, H., Hasegawa, K., Kida, H., Kijima, T., Morimoto, S., Fujiki, F., Tsuboi, A., Morii, E., Morita, S., Sakamoto, J., Kumanogoh, A., Oka, Y., Okumura, M., and Sugiyama, H. (2018) WT1 peptide-based immunotherapy for advanced thymic epithelial malignancies. Int J Cancer 142, 2375-2382
237. Liu, H., Zha, Y., Choudhury, N., Malnassy, G., Fulton, N., Green, M., Park, J. H., Nakamura, Y., Larson, R. A., Salazar, A. M., Odenike, O., Gajewski, T. F., and Stock, W. (2018) WT1 peptide vaccine in Montanide in contrast to poly ICLC, is able to induce WT1-specific immune response with TCR clonal enrichment in myeloid leukemia. Exp Hematol Oncol 7, 1
238. Nishida, S., Ishikawa, T., Egawa, S., Koido, S., Yanagimoto, H., Ishii, J., Kanno, Y., Kokura, S., Yasuda, H., Oba, M. S., Sato, M., Morimoto, S., Fujiki, F., Eguchi, H., Nagano, H., Kumanogoh, A., Unno, M., Kon, M., Shimada, H., Ito, K., Homma, S., Oka, Y., Morita, S., and Sugiyama, H. (2018) Combination Gemcitabine and WT1 Peptide Vaccination Improves Progression-Free Survival in Advanced Pancreatic Ductal Adenocarcinoma: A Phase II Randomized Study. Cancer Immunol Res 6, 320-331
239. Maslak, P. G., Dao, T., Bernal, Y., Chanel, S. M., Zhang, R., Frattini, M., Rosenblat, T., Jurcic, J. G., Brentjens, R. J., Arcila, M. E., Rampal, R., Park, J. H., Douer, D., Katz, L., Sarlis, N., Tallman, M. S., and Scheinberg, D. A. (2018) Phase 2 trial of a multivalent WT1 peptide vaccine (galinpepimut-S) in acute myeloid leukemia. Blood Adv 2, 224-234
240. Hirabayashi, K., Yanagisawa, R., Saito, S., Higuchi, Y., Koya, T., Sano, K., Koido, S., Okamoto, M., Sugiyama, H., Nakazawa, Y., and Shimodaira, S. (2018) Feasibility and Immune Response of WT1 Peptide Vaccination in Combination with OK-432 for Paediatric Solid Tumors. Anticancer Res 38, 2227-2234
241. Kida, A., Mizukoshi, E., Tamai, T., Terashima, T., Kitahara, M., Arai, K., Yamashita, T., Fushimi, K., Honda, M., and Kaneko, S. (2018) Immune responses against tumour-associated antigen-derived cytotoxic T lymphocyte epitopes in cholangiocarcinoma patients. Liver Int 38, 2040-2050
242. Ogasawara, M., Miyashita, M., and Ota, S. (2018) Vaccination of Urological Cancer Patients With WT1 Peptide-Pulsed Dendritic Cells in Combination With Molecular Targeted Therapy or Conventional Chemotherapy Induces Immunological and Clinical Responses. Ther Apher Dial 22, 266-277
243. Deicher, A., Andersson, R., Tingstedt, B., Lindell, G., Bauden, M., and Ansari, D. (2018) Targeting dendritic cells in pancreatic ductal adenocarcinoma. Cancer Cell Int 18, 85
244. Matsuda, T., Takeuchi, H., Sakurai, T., Mayanagi, S., Booka, E., Fujita, T., Higuchi, H., Taguchi, J., Hamamoto, Y., Takaishi, H., Kawakubo, H., Okamoto, M., Sunamura, M., Kawakami, Y., and Kitagawa, Y. (2018) Pilot study of WT1 peptide-pulsed dendritic cell vaccination with docetaxel in esophageal cancer. Oncol Lett 16, 1348-1356
245. Lu, X., Liu, J., Cui, P., Liu, T., Piao, C., Xu, X., Zhang, Q., Xiao, M., Liu, X., Wang, Y., and Yang, L. (2018) Co-inhibition of TIGIT, PD1, and Tim3 reverses dysfunction of Wilms tumor protein-1 (WT1)-specific CD8+ T lymphocytes after dendritic cell vaccination in gastric cancer. Am J Cancer Res 8, 1564-1575
246. Hanada, S., Tsuruta, T., Haraguchi, K., Okamoto, M., Sugiyama, H., and Koido, S. (2019) Long-term survival of pancreatic cancer patients treated with multimodal therapy combined with WT1-targeted dendritic cell vaccines. Hum Vaccin Immunother 15, 397-406
247. Zhang, W., Lu, X., Cui, P., Piao, C., Xiao, M., Liu, X., Wang, Y., Wu, X., Liu, J., and Yang, L. (2019) Phase I/II clinical trial of a Wilms' tumor 1-targeted dendritic cell vaccination-based immunotherapy in patients with advanced cancer. Cancer Immunol Immunother 68, 121-130
248. Xiang, S. D., Wilson, K. L., Goubier, A., Heyerick, A., and Plebanski, M. (2018) Design of Peptide-Based Nanovaccines Targeting Leading Antigens From Gynecological Cancers to Induce HLA-A2.1 Restricted CD8(+) T Cell Responses. Front Immunol 9, 2968
249. Kitagawa, K., Gonoi, R., Tatsumi, M., Kadowaki, M., Katayama, T., Hashii, Y., Fujisawa, M., and Shirakawa, T. (2019) Preclinical Development of a WT1 Oral Cancer Vaccine Using a Bacterial Vector to Treat Castration-Resistant Prostate Cancer. Mol Cancer Ther 18, 980-990
250. Comoli, P., Basso, S., Riva, G., Barozzi, P., Guido, I., Gurrado, A., Quartuccio, G., Rubert, L., Lagreca, I., Vallerini, D., Forghieri, F., Morselli, M., Bresciani, P., Cuoghi, A., Paolini, A., Colaci, E., Marasca, R., Cuneo, A., lughetti, L., Trenti, T., Narni, F., Foa, R., Zecca, M., Luppi, M., and Potenza, L. (2017) BCR-ABL-specific T-cell therapy in Ph+ALL patients on tyrosine-kinase inhibitors. Blood 129, 582-586
251. Ueda, N., Zhang, R., Tatsumi, M., Liu, T. Y., Kitayama, S., Yasui, Y., Sugai, S., Iwama, T., Senju, S., Okada, S., Nakatsura, T., Kuzushima, K., Kiyoi, H., Naoe, T., Kaneko, S., and Uemura, Y. (2018) BCR-ABL-specific CD4(+) T-helper cells promote the priming of antigen-specific cytotoxic T cells via dendritic cells. Cell Mol Immunol 15, 15-26
252. Rojas, J. M., Knight, K., Wang, L., and Clark, R. E. (2007) Clinical evaluation of BCR-ABL peptide immunisation in chronic myeloid leukaemia: results of the EPIC study. Leukemia 21, 2287-2295
253. Scheich, F., Duyster, J., Peschel, C., and Bernhard, H. (2007) The immunogenicity of Bcr-Abl expressing dendritic cells is dependent on the Bcr-Abl kinase activity and dominated by Bcr-Abl regulated antigens. Blood 110, 2556-2560
254. Brauer, K. M., Werth, D., von Schwarzenberg, K., Bringmann, A., Kanz, L., Grunebach, F., and Brossart, P. (2007) BCR-ABL activity is critical for the immunogenicity of chronic myelogenous leukemia cells. Cancer Res 67, 5489-5497
255. Kislin, K. L., Marron, M. T., Li, G., Graner, M. W., and Katsanis, E. (2007) Chaperone-rich cell lysate embedded with BCR-ABL peptide demonstrates enhanced anti-tumor activity against a murine BCR-ABL positive leukemia. FASEB J 21, 2173-2184
256. Bornhauser, M., Thiede, C., Babatz, J., Schetelig, J., Illmer, T., Kiani, A., Platzbecker, U., Herr, W., Rieber, E. P., Ehninger, G., and Schmitz, M. (2006) Infusion of bcr/abl peptide-reactive donor T cells to achieve molecular remission of chronic myeloid leukemia after CD34+ selected allogeneic hematopoietic cell transplantation. Leukemia 20, 2055-2057
257. Zeng, Y., Graner, M. W., Thompson, S., Marron, M., and Katsanis, E. (2005) Induction of BCR-ABL-specific immunity following vaccination with chaperone-rich cell lysates derived from BCR-ABL+ tumor cells. Blood 105, 2016-2022
258. Sun, J. Y., Senitzer, D., Forman, S. J., Chatterjee, S., and Wong, K. K., Jr. (2003) Identification of new MHC-restriction elements for presentation of the p210(BCR-ABL) fusion region to human cytotoxic T lymphocytes. Cancer Immunol Immunother 52, 761-770
259. Zorn, E., Orsini, E., Wu, C. J., Stein, B., Chillemi, A., Canning, C., Alyea, E. P., Soiffer, R. J., and Ritz, J. (2001) A CD4+ T cell clone selected from a CML patient after donor lymphocyte infusion recognizes BCR-ABL breakpoint peptides but not tumor cells. Transplantation 71, 1131-1137
260. Westermann, J., Kopp, J., Korner, I., Richter, G., Qin, Z., Blankenstein, T., Dorken, B., and Pezzutto, A. (2000) Bcr/abl+ autologous dendritic cells for vaccination in chronic myeloid leukemia. Bone Marrow Transplant 25 Suppl 2, S46-49
261. Tanaka, Y., Takahashi, T., Nieda, M., Masuda, S., Kashiwase, K., Ogawa, S., Chiba, S., Juji, T., and Hirai, H. (2000) Generation of HLA-DRB1*1501-restricted p190 minor bcr-abl (ela2)-specific CD4+ T lymphocytes. Br J Haematol 109, 435-437
262. Nieda, M., Nicol, A., Kikuchi, A., Kashiwase, K., Taylor, K., Suzuki, K., Tadokoro, K., and Juji, T. (1998) Dendritic cells stimulate the expansion of bcr-abl specific CD8+ T cells with cytotoxic activity against leukemic cells from patients with chronic myeloid leukemia. Blood 91, 977-983
263. Mannering, S. I., McKenzie, J. L., Fearnley, D. B., and Hart, D. N. (1997) HLA-DR1-restricted bcr-abl (b3a2)-specific CD4+ T lymphocytes respond to dendritic cells pulsed with b3a2 peptide and antigen-presenting cells exposed to b3a2 containing cell lysates. Blood 90, 290-297
264. Smit, W. M., Rijnbeek, M., van Bergen, C. A., de Paus, R. A., Vervenne, H. A., van de Keur, M., Willemze, R., and Falkenburg, J. H. (1997) Generation of dendritic cells expressing bcr-abl from CD34-positive chronic myeloid leukemia precursor cells. Hum Immunol 53, 216-223
265. Smith, M. C., Pendleton, C. D., Maher, V. E., Kelley, M. J., Carbone, D. P., and Berzofsky, J. A. (1997) Oncogenic mutations in ras create HLA-A2.1 binding peptides but affect their extracellular antigen processing. Int Immunol 9, 1085-1093
266. Ding, L., Lalani, E. N., Reddish, M., Koganty, R., Wong, T., Samuel, J., Yacyshyn, M. B., Meikle, A., Fung, P. Y., Taylor-Papadimitriou, J., and et al. (1993) Immunogenicity of synthetic peptides related to the core peptide sequence encoded by the human MUC1 mucin gene: effect of immunization on the growth of murine mammary adenocarcinoma cells transfected with the human MUC1 gene. Cancer Immunol Immunother 36, 9-17
267. Brossart, P., Heinrich, K. S., Stuhler, G., Behnke, L., Reichardt, V. L., Stevanovic, S., Muhm, A., Rammensee, H. G., Kanz, L., and Brugger, W. (1999) Identification of HLA-A2-restricted T-cell epitopes derived from the MUC1 tumor antigen for broadly applicable vaccine therapies. Blood 93, 4309-4317
268. Koido, S., Kashiwaba, M., Chen, D., Gendler, S., Kufe, D., and Gong, J. (2000) Induction of antitumor immunity by vaccination of dendritic cells transfected with MUC1 RNA. J Immunol 165, 5713-5719
269. Gong, J., Apostolopoulos, V., Chen, D., Chen, H., Koido, S., Gendler, S. J., McKenzie, I. F., and Kufe, D. (2000) Selection and characterization of MUC1-specific CD8+ T cells from MUC1 transgenic mice immunized with dendritic-carcinoma fusion cells. Immunology 101, 316-324
270. Apostolopoulos, V., Pietersz, G. A., and McKenzie, I. F. (1999) MUC1 and breast cancer. Curr Opin Mol Ther 1, 98-103
271. Soares, M. M., Mehta, V., and Finn, O. J. (2001) Three different vaccines based on the 140-amino acid MUC1 peptide with seven tandemly repeated tumor-specific epitopes elicit distinct immune effector mechanisms in wild-type versus MUC1-transgenic mice with different potential for tumor rejection. J Immunol 166, 6555-6563
272. Tanaka, Y., Koido, S., Chen, D., Gendler, S. J., Kufe, D., and Gong, J. (2001) Vaccination with allogeneic dendritic cells fused to carcinoma cells induces antitumor immunity in MUC1 transgenic mice. Clin Immunol 101, 192-200
273. Pecher, G., Spahn, G., Schirrmann, T., Kulbe, H., Ziegner, M., Schenk, J. A., and Sandig, V. (2001) Mucin gene (MUC1) transfer into human dendritic cells by cationic liposomes and recombinant adenovirus. Anticancer Res 21, 2591-2596
274. Trevor, K. T., Hersh, E. M., Brailey, J., Balloul, J. M., and Acres, B. (2001) Transduction of human dendritic cells with a recombinant modified vaccinia Ankara virus encoding MUC1 and IL-2. Cancer Immunol Immunother 50, 397-407
275. Pecher, G., Haring, A., Kaiser, L., and Thiel, E. (2002) Mucin gene (MUC1) transfected dendritic cells as vaccine: results of a phase I/II clinical trial. Cancer Immunol Immunother 51, 669-673
276. Xia, J., Tanaka, Y., Koido, S., Liu, C., Mukherjee, P., Gendler, S. J., and Gong, J. (2003) Prevention of spontaneous breast carcinoma by prophylactic vaccination with dendritic/tumor fusion cells. J Immunol 170, 1980-1986
277. Kontani, K., Taguchi, O., Ozaki, Y., Hanaoka, J., Sawai, S., Inoue, S., Abe, H., Hanasawa, K., and Fujino, S. (2003) Dendritic cell vaccine immunotherapy of cancer targeting MUC1 mucin. Int J Mol Med 12, 493-502
278. Apostolopoulos, V., Pouniotis, D. S., van Maanen, P. J., Andriessen, R. W., Lodding, J., Xing, P. X., McKenzie, I. F., Loveland, B. E., and Pietersz, G. A. (2006) Delivery of tumor associated antigens to antigen presenting cells using penetratin induces potent immune responses. Vaccine 24, 3191-3202
279. Ryan, S. O., Vlad, A. M., Islam, K., Gariepy, J., and Finn, O. J. (2009) Tumor-associated MUC1 glycopeptide epitopes are not subject to self-tolerance and improve responses to MUC1 peptide epitopes in MUC1 transgenic mice. Biol Chem 390, 611-618
280. Wang, K., Zhou, Q., Guo, A. L., Xu, C. R., An, S. J., and Wu, Y. L. (2009) An autologous therapeutic dendritic cell vaccine transfected with total lung carcinoma RNA stimulates cytotoxic T lymphocyte responses against non-small cell lung cancer. Immunol Invest 38, 665-680
281. Vasir, B., Zarwan, C., Ahmad, R., Crawford, K. D., Rajabi, H., Matsuoka, K., Rosenblatt, J., Wu, Z., Mills, H., Kufe, D., and Avigan, D. (2012) Induction of antitumor immunity ex vivo using dendritic cells transduced with fowl pox vector expressing MUC1, CEA, and a triad of costimulatory molecules (rF-PANVAC). J Immunother 35, 555-569
282. Acres, B., Lacoste, G., and Limacher, J. M. (2017) Targeted Immunotherapy Designed to Treat MUC1-Expressing Solid Tumour. Curr Top Microbiol Immunol 405, 79-97
283. Sui, D., Ma, L., Li, M., Shao, W., Du, H., Li, K., Li, Z., and Li, Z. (2016) Mucin 1 and poly I:C activates dendritic cells and effectively eradicates pituitary tumors as a prophylactic vaccine. Mol Med Rep 13, 3675-3683
284. Glaffig, M., Stergiou, N., Schmitt, E., and Kunz, H. (2017) Immunogenicity of a Fully Synthetic MUC1 Glycopeptide Antitumor Vaccine Enhanced by Poly(I:C) as a TLR3-Activating Adjuvant. ChemMedChem 12, 722-727
285. Scheid, E., Major, P., Bergeron, A., Finn, O. J., Salter, R. D., Eady, R., Yassine-Diab, B., Favre, D., Peretz, Y., Landry, C., Hotte, S., Mukherjee, S. D., Dekaban, G. A., Fink, C., Foster, P. J., Gaudet, J., Gariepy, J., Sekaly, R. P., Lacombe, L., Fradet, Y., and Foley, R. (2016) Tn-MUC1 DC Vaccination of Rhesus Macaques and a Phase I/II Trial in Patients with Nonmetastatic Castrate-Resistant Prostate Cancer. Cancer Immunol Res 4, 881-892
286. Wang, D., Huang, X. F., Hong, B., Song, X. T., Hu, L., Jiang, M., Zhang, B., Ning, H., Li, Y., Xu, C., Lou, X., Li, B., Yu, Z., Hu, J., Chen, J., Yang, F., Gao, H., Ding, G., Liao, L., Rollins, L., Jones, L., Chen, S. Y., and Chen, H. (2018) Efficacy of intracellular immune checkpoint-silenced DC vaccine. JCI Insight 3
287. Yotnda, P., Garcia, F., Peuchmaur, M., Grandchamp, B., Duval, M., Lemonnier, F., Vilmer, E., and Langlade-Demoyen, P. (1998) Cytotoxic T cell response against the chimeric ETV6-AML1 protein in childhood acute lymphoblastic leukemia. J Clin Invest 102, 455-462
288. Redchenko, I. V., and Rickinson, A. B. (1999) Accessing Epstein-Barr virus-specific T-cell memory with peptide-loaded dendritic cells. J Virol 73, 334-342
289. Ranieri, E., Herr, W., Gambotto, A., Olson, W., Rowe, D., Robbins, P. D., Kierstead, L. S., Watkins, S. C., Gesualdo, L., and Storkus, W. J. (1999) Dendritic cells transduced with an adenovirus vector encoding Epstein-Barr virus latent membrane protein 2B: a new modality for vaccination. J Virol 73, 10416-10425
290. Su, Z., Peluso, M. V., Raffegerst, S. H., Schendel, D. J., and Roskrow, M. A. (2001) The generation of LMP2a-specific cytotoxic T lymphocytes for the treatment of patients with Epstein-Barr virus-positive Hodgkin disease. Eur J Immunol 31, 947-958
291. Leen, A., Meij, P., Redchenko, I., Middeldorp, J., Bloemena, E., Rickinson, A., and Blake, N. (2001) Differential immunogenicity of Epstein-Barr virus latent-cycle proteins for human CD4(+) T-helper 1 responses. J Virol 75, 8649-8659
292. Bollard, C. M., Straathof, K. C., Huls, M. H., Leen, A., Lacuesta, K., Davis, A., Gottschalk, S., Brenner, M. K., Heslop, H. E., and Rooney, C. M. (2004) The generation and characterization of LMP2-specific CTLs for use as adoptive transfer from patients with relapsed EBV-positive Hodgkin disease. J Immunother 27, 317-327
293. Chia, W. K., Wang, W. W., Teo, M., Tai, W. M., Lim, W. T., Tan, E. H., Leong, S. S., Sun, L., Chen, J. J., Gottschalk, S., and Toh, H. C. (2012) A phase II study evaluating the safety and efficacy of an adenovirus-DeltaLMP1-LMP2 transduced dendritic cell vaccine in patients with advanced metastatic nasopharyngeal carcinoma. Ann Oncol 23, 997-1005
294. Hong, B., Peng, G., Berry, L., Gottschalk, S., Jung, J. U., Chen, S. Y., and Huang, X. F. (2012) Generating CTLs against the subdominant EBV LMP antigens by transient expression of an A20 inhibitor with EBV LMP proteins in human DCs. Gene Ther 19, 818-827
295. Si, Y., Deng, Z., Lan, G., Du, H., Wang, Y., Si, J., Wei, J., Weng, J., Qin, Y., Huang, B., Yang, Y., and Qin, Y. (2016) The Safety and Immunological Effects of rAd5-EBV-LMP2 Vaccine in Nasopharyngeal Carcinoma Patients: A Phase I Clinical Trial and Two-Year Follow-Up. Chem Pharm Bull (Tokyo) 64, 1118-1123
296. Tilkin, A. F., Lubin, R., Soussi, T., Lazar, V., Janin, N., Mathieu, M. C., Lefrere, I., Carlu, C., Roy, M., Kayibanda, M., and et al. (1995) Primary proliferative T cell response to wild-type p53 protein in patients with breast cancer. Eur J Immunol 25, 1765-1769
297. Roth, J., Dittmer, D., Rea, D., Tartaglia, J., Paoletti, E., and Levine, A. J. (1996) p53 as a target for cancer vaccines: recombinant canarypox virus vectors expressing p53 protect mice against lethal tumor cell challenge. Proc Natl Acad Sci USA 93, 4781-4786
298. Zusman, I., Zusman, R., Gurevich, P., Korol, D., and Tendler, Y. (1996) High tumor-preventive effects of polyclonal IgG generated against p53 tumor-associated protein obtained from benign-tumor bearing rats. Oncol Rep 3, 975-979
299. Takahara, A., Koido, S., Ito, M., Nagasaki, E., Sagawa, Y., Iwamoto, T., Komita, H., Ochi, T., Fujiwara, H., Yasukawa, M., Mineno, J., Shiku, H., Nishida, S., Sugiyama, H., Tajiri, H., and Homma, S. (2011) Gemcitabine enhances Wilms' tumor gene WT1 expression and sensitizes human pancreatic cancer cells with WT1-specific T-cell-mediated antitumor immune response. Cancer Immunol Immunother 60, 1289-1297
300. Kwon, Y. R., Son, M. J., Kim, H. J., and Kim, Y. J. (2012) Reactivation of Silenced WT1 Transgene by Hypomethylating Agents—Implications for in vitro Modeling of Chemoimmunotherapy. Immune Netw 12, 58-65
301. Kitagawa, K., Oda, T., Saito, H., Araki, A., Gonoi, R., Shigemura, K., Hashii, Y., Katayama, T., Fujisawa, M., and Shirakawa, T. (2017) Development of oral cancer vaccine using recombinant Bifidobacterium displaying Wilms' tumor 1 protein. Cancer Immunol Immunother 66, 787-798

1. A method of augmenting immune stimulatory activity of dendritic cells comprising:
a) providing a dendritic cell population;
b) treating said dendritic cell population with cannabidiol; and
c) suppressing expression and/or activity of NR2F6 in said dendritic cells.

2. The method of claim 1, wherein said immune stimulatory activity is ability of dendritic cells to activate T cells.

3. The method of claim 2, wherein said T cell activation is proliferation.

4. The method of claim 2, wherein said T cell activation is cytokine production.

5. The method of claim 2, wherein said T cell activation is ability to convert to memory cells.

6. The method of claim 2, wherein said T cell activation is acquisition of cytotoxic activity.

7. The method of claim 2, wherein said T cell activation is ability to activate antibody production from B cells.

8. The method of claim 2, wherein said T cell activation is ability to induce isotype switching.

9. The method of claim 2, wherein said T cell activation is ability to induce maturation of dendritic cells.

10. The method of claim 2, wherein said T cell activation is ability to induce NK cells to possess an enhancement of cytotoxic activity.

11. The method of claim 1, wherein said dendritic cells are myeloid derived dendritic cells.

12. The method of claim 1, wherein said dendritic cells are lymphoid derived dendritic cells.

13. The method of claim 1, wherein said cannabidiol possesses anti-inflammatory properties.

14. The method of claim 1, wherein said dendritic cell is further activated with a toll like receptor agonist.

15. The method of claim 1, wherein suppression of NR2F6 is achieved by administration of an antisense oligonucleotide molecule capable of suppressing expression of NR2F6.

16. The method of claim 15, wherein suppression of said NR2F6 is mediated by activation of RNAse H.

17. The method of claim 1, wherein said suppression of NR2F6 is achieved by treatment of said dendritic cells with nucleic acids capable of inducing the processes of RNA interference.

18. The method of claim 17, wherein said nucleic acids capable of inducing the process of RNA interference are short double stranded RNA.
